(19) United States
(12) Patent Application Publication
(10) Pub. No.: US 2024/0190951 A1
(43) Pub. Date: Jun. 13, 2024

(54) ANTI-TSLP ANTIBODY COMPOSITIONS AND USES THEREOF

(71) Applicant: AMGEN INC., Thousand Oaks, CA (US)

(72) Inventors: Hao Zhang, Lexington, MA (US); Alla Polozova, North Kingstown, RI (US); Kelly Fitzpatrick, Winchester, MA (US); Kristin Abrams, Reading, MA (US); Dong Xiang, Thousand Oaks, CA (US); Marisa K. Joubert, Newbury Park, CA (US)

(21) Appl. No.: 18/287,593

(22) PCT Filed: Apr. 22, 2022
(86) PCT No.: PCT/US22/25994

§ 371 Date: Oct. 19, 2023

Related U.S. Application Data
(60) Provisional application No. 63/178,938, filed on Apr. 23, 2021.

(51) Int. Cl. C07K 16/24; A61K 39/00; G01N 33/68

(52) U.S. Cl. CPC C07K 16/244; G01N 33/6845; G01N 33/6854; A61K 39/00; C07K 2317/21; C07K 2317/41; G01N 2500/10

(57) ABSTRACT

The present application relates, in general, to compositions comprising anti-TSLP antibody tezepelumab and derivatives thereof having antibody quality attributes.

### CROSS-REFERENCE TO RELATED APPLICATIONS

The present application claims the priority benefit of U.S. Provisional Patent Application No. 63/178,938, filed Apr. 23, 2021, hereby incorporated by reference in its entirety.

### FIELD OF THE DISCLOSURE

The present application relates, in general, to compositions comprising anti-TSLP antibody tezepelumab and derivatives of thereof comprising antibody quality attributes.

### BACKGROUND OF THE DISCLOSURE

Thymic stromal lymphopoietin (TSLP), an epithelial cell-derived cytokine produced in response to environmental and pro-inflammatory stimuli, leads to the activation of multiple inflammatory cells and downstream pathways (Soumelis et al. Nat Immunol 2002; 3:673-80; Allakhverdi et al. J Exp Med 2007; 204:253-8). TSLP is increased in the airways of patients with asthma and correlates with Th2 cytokine and chemokine expression (Shikotra et al. J Allergy Clin Immunol 2012; 129:104-11 e1-9) and disease severity (Ying et al. J Immunol 2005; 174:8183-90; Ying et al. J Immunol 2008; 181:2790-8). While TSLP is central to the regulation of Th2 immunity, it may also play a key role in other pathways of inflammation and therefore be relevant to multiple asthma phenotypes.

Tezepelumab is a human immunoglobulin G2 (IgG2) monoclonal antibody (mAb) that binds to TSLP, preventing its interaction with the TSLP receptor complex. It will be appreciated that tezepelumab is a heterotetramer comprising two heavy chains and two light chains, and comprising two binding site to TSLP. A proof-of-concept study in patients with mild, atopic asthma, demonstrated that tezepelumab inhibited the early and late asthmatic responses and suppressed biomarkers of Th2 inflammation following inhaled allergen challenge (Gauvreau, et al. N Engl J Med 2014; 370:2102-10).

### SUMMARY

Monitoring of antibody therapeutics in formulation over time is important to determine storage conditions that reduce any breakdown of the therapeutic and maintain the integrity of the product. The present disclosure provides a study of attributes of an anti-TSLP antibody that can change over time during manufacturing and storage, including attributes that can be beneficial or detrimental to antibody tolerability and/or potency.

In one aspect, the disclosure provides a composition comprising tezepelumab and one or more tezepelumab derivatives, wherein the one or more tezepelumab derivatives comprise an isomerization derivative, and wherein the amount of the isomerization derivative in the composition is less than about 30%, wherein tezepelumab comprises (A) a light chain variable domain comprising: (i) a light chain CDR1 amino acid sequence set out in SEQ ID NO:3; (ii) a light chain CDR2 amino acid sequence set out in SEQ ID NO: 4; and (iii) a light chain CDR3 amino acid sequence set out in SEQ ID NO:5; and (B) a heavy chain variable domain comprising: (i) a heavy chain CDR1 amino acid sequence set out in SEQ ID NO:6; (ii) a heavy chain CDR2 amino acid sequence set out in SEQ ID NO:7 and (iii) a heavy chain CDR3 amino acid sequence set out in SEQ ID NO:8.

In various embodiments, the amount of the isomerization derivative in the composition is less than about 30%, 25%, 20%, 15%, 10%, 5%, 4%, 3%, 2% or 1%. In various embodiments, the amount of the isomerization derivative in the composition is from about 0.5% to about 13%. In various embodiments, the amount of isomerization derivative in the composition is from about 1% to 12%, 2% to 10% or about 4% to 7%. In various embodiments, the isomerization derivative comprises a modification in the heavy chain or light chain complementarity determining region (CDR). In various embodiments, isomerization derivative comprises a change at heavy chain CDR residue D54 of SEQ ID NO: 7, and/or light chain CDR residue D49, D50 or D52 of SEQ ID NO: 4, in either or both variable region chains. In various embodiments, the isomerization derivative comprises isomerization at D54 in an amount of less than about 5%. In various embodiments, the isomerization derivative comprises isomerization at D54 in an amount of less than about 4%, 3%, 2% or 1%. In various embodiments, the isomerization derivative comprises isomerization at one or more of residues D49, D50 or D52 of SEQ ID NO: 4 in an amount of less than about 26%. In various embodiments, the isomerization derivative comprises isomerization at one or more of residues D49, D50 or D52 of SEQ ID NO: 4 in an amount of less than about 25%, 20%, 18%, 15%, 13%, 10%, 7%, 5%, 3% or 2%. In various embodiments, the isomerization derivative is isoaspartic acid (isoAsp), cyclic aspartate (cAsp), succinimide or an isomerization intermediate. In various embodiments, the isomerization derivative is isoaspartic acid (isoAsp) or cyclic aspartate (cAsp). In various embodiments, the amount of the isomerization derivative in the composition is determined by reduced peptide mapping. In various embodiments, the tezepelumab and tezepelumab derivatives have a greater potency and/or tolerability than a composition comprising greater than 30% of the isomerization derivative, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 30% of the isomerization derivative, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

A composition comprising tezepelumab and one or more tezepelumab derivatives, wherein the one or more tezepelumab derivatives comprise a deamidation derivative, and wherein the amount of the deamidation derivative in the composition is less than about 15%, wherein tezepelumab comprises (A) a light chain variable domain comprising: (i) a light chain CDR1 amino acid sequence set out in SEQ ID NO:3; (ii) a light chain CDR2 amino acid sequence set out in SEQ ID NO: 4; and (iii) a light chain CDR3 amino acid sequence set out in SEQ ID NO:5; and (B) a heavy chain variable domain comprising: (i) a heavy chain CDR1 amino acid sequence set out in SEQ ID NO:6; (ii) a heavy chain CDR2 amino acid sequence set out in SEQ ID NO:7 and (iii) a heavy chain CDR3 amino acid sequence set out in SEQ ID NO:8. In various embodiments, the amount of the deamidation derivative in the composition is less than about 15%, about 10%, about 7%, about 5%, about 4%, about 3%, about 2% or about 1%. In various embodiments, the amount of the deamidation derivative in the composition is from about 0.5% to about 13%. In various embodiments, the amount of the deamidation derivative in the composition is between about 0.5%-10%, about 1% to 8%, about 2% to 7% or about 3% to 6%. In various embodiments, the deamidation derivative comprises deamidated asparagine residue N25/N26 in LCDR1 set out in SEQ ID NO: 3, residue N316 in the heavy chain set out in SEQ ID NO: 13, and/or residue N385/390 in the heavy chain set out in SEQ ID NO: 13. In various embodiments, the deamidation derivative comprises deamidation at N25/N26 in an amount of less than about 3%. In various embodiments, the deamidation derivative comprises deamidation at one or more of N316, and/or N385/390 in an amount of less than about 13%. In various embodiments, the deamidation derivative is deamidated asparagine or a deamidation intermediate. In various embodiments, the amount of the deamidation derivative in the composition is determined by reduced peptide mapping. In various embodiments, the tezepelumab and tezepelumab derivatives have a greater potency and/or tolerability than a composition comprising greater than 15% of the deamidation derivative, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 15% of the deamidation derivative, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

The disclosure also provides a composition comprising tezepelumab and one or more tezepelumab derivatives, wherein the one or more tezepelumab derivatives comprise an oxidation derivative, and wherein the amount of the oxidation derivative in the composition is less than about 7%, wherein tezepelumab comprises (A) a light chain variable domain comprising: (i) a light chain CDR1 amino acid sequence set out in SEQ ID NO:3; (ii) a light chain CDR2 amino acid sequence set out in SEQ ID NO: 4; and (iii) a light chain CDR3 amino acid sequence set out in SEQ ID NO:5; and (B) a heavy chain variable domain comprising: (i) a heavy chain CDR1 amino acid sequence set out in SEQ ID NO:6; (ii) a heavy chain CDR2 amino acid sequence set out in SEQ ID NO:7 and (iii) a heavy chain CDR3 amino acid sequence set out in SEQ ID NO:8.

In various embodiments, the amount of the oxidation derivative is less than about 6%, about 5%, about 4%, about 3%, about 2% or about 1%. In various embodiments, the amount of the oxidation derivative in the composition is between about 0.4% to about 7%, about 1% to about 6%, about 2% to about 5% or about 0.4% to about 4%. In various embodiments, the oxidation derivative comprises oxidation at one or more of heavy chain methionine residue M34 of HCDR1 set out in SEQ ID NO: 6, or residue M253 or M359 in the heavy chain constant region set out in SEQ ID NO: 13, or heavy chain tryptophan residue W52 in HCDR2 set out in SEQ ID NO: 7, W90 of LCDR3 set out in SEQ ID NO: 5, or W102 in HCDR3 set out in SEQ ID NO: 8, in either or both heavy chains. In various embodiments, the oxidation derivative comprises oxidation at one or more of heavy chain methionine residues M34, M253, M359, in either or both heavy chains, optionally wherein the oxidation is in an amount of less than about 7%. In various embodiments, the oxidation derivative comprises oxidation at one or more of tryptophan residues W52, W90, or W102 in either or both heavy chains, optionally wherein the oxidation is in an amount of less than about 3%. In various embodiments, the amount of the oxidation derivative in the composition is determined by reduced peptide mapping. In various embodiments, the tezepelumab and tezepelumab derivatives have a greater potency and/or tolerability than a composition comprising greater than 7% of the oxidation derivative, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 7% of the oxidation derivative, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

In another embodiment, the disclosure provides a composition comprising tezepelumab and one or more tezepelumab derivatives, wherein the one or more tezepelumab derivatives comprise a glycosylation derivative, and wherein the amount of the glycosylation derivative in the composition is less than about 40%, wherein tezepelumab comprises (A) a light chain variable domain comprising: (i) a light chain CDR1 amino acid sequence set out in SEQ ID NO:3; (ii) a light chain CDR2 amino acid sequence set out in SEQ ID NO: 4; and (iii) a light chain CDR3 amino acid sequence set out in SEQ ID NO:5; and (B) a heavy chain variable domain comprising: (i) a heavy chain CDR1 amino acid sequence set out in SEQ ID NO:6; (ii) a heavy chain CDR2 amino acid sequence set out in SEQ ID NO:7 and (iii) a heavy chain CDR3 amino acid sequence set out in SEQ ID NO:8.

In various embodiments, the amount of glycosylation derivative in the composition is less than about 35%, about 30%, about 25%, about 20%, about 15%, about 10%, about 9%, about 8% about 7%, about 6%, or about 5%. In various embodiments, the amount of glycosylation derivative in the composition is between about 1% to about 35%, about 3% to about 30%, about 5% to about 25%, about 10% to about 20%. In various embodiments, the glycosylation derivative comprises alteration of tezepelumab glycosylation on residue N298 of SEQ ID NO: 13, on one or both heavy chains. In various embodiments, the glycosylation derivative comprises afucosylation or alteration of glycosylation of tezepelumab to high mannose moieties or galactosyl moieties. In various embodiments, the glycosylation derivative comprises afucosylated derivative in an amount of less than about 5%. In various embodiments, the glycosylation derivative comprises galactosyl moieties in an amount of less than about 30%. In various embodiments, the glycosylation derivative comprises high mannose moieties in an amount of less than about 5%. In various embodiments, the amount of the glycosylation derivative in the composition is determined by glycan map method. In various embodiments, the tezepelumab and tezepelumab derivatives have a greater potency and/or tolerability than a composition comprising greater than 40% of the glycosylation derivatives, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 40% of the glycosylation derivatives, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

Glycosylation derivatives can also be associated with effector function and antibody clearance (it will be appreciated that less antibody clearance can be indicative of longer half-life; as such an antibody or antibody composition having “less clearance” than a reference antibody or antibody composition will be understood to refer to a numerically longer half life than the reference antibody or antibody composition). In various embodiments, the tezepelumab and tezepelumab derivatives have less antibody clearance and/or greater tolerability than a composition comprising greater than about 15%, about 13%, about 11%, about 8% or about 6% high mannose glycosylation derivatives. In various embodiments, the tezepelumab and tezepelumab derivatives have less antibody clearance and/or greater tolerability than a composition comprising greater than about 25%, about 23% (e.g., about 23.1%), about 21%, about 18%, about 15%, about 13%, about 11%, about 8%, about 6%, or about 5% high mannose glycosylation derivatives. In various embodiments, the tezepelumab and tezepelumab derivatives have less antibody clearance and/or greater tolerability than a composition comprising greater than about 23.1% high mannose glycosylation derivatives. In various embodiments, an increase of high mannose species from 5% to 23.1% results in no more than 1.7% or 10% increase in clearance of tezepelumab and tezepelumab derivatives.

Also provided is a composition comprising tezepelumab and one or more disulfide isoform derivatives thereof, wherein the one or more disulfide isoform derivatives comprise an IgG2-B isoform and/or an IgG2-A/B isoform, and wherein the amount of the disulfide isoform in the composition is less than about 75%. In various embodiments, the amount of disulfide isoform in the composition is less than about 70%, about 65, about 60%, about 55%, about 50%, about 45%, about 40%, about 35%, about 30%, about 25%, about 20%, about 15%, about 10%, about 5%, about 4%, about 3%, about 2% or about 1%.

In various embodiments, the one or more disulfide isoform derivatives comprise an IgG2-B isoform. In various embodiments, the amount of the IgG2-B isoform is less than about 5%. In various embodiments, the one or more disulfide isoform derivatives comprise an IgG2-A/B isoform. In various embodiments, the amount of the IgG2-A/B isoform in the composition is less than about 75%. In various embodiments, the amount of the IgG2-A/B isoform in the composition is from about 38% to about 43%. In various embodiments, the amount of disulfide isoform derivatives in the composition is determined by non-reduced reversed phase high performance liquid chromatography (RP-HPLC).

Also contemplated is a composition comprising tezepelumab and one or more tezepelumab derivatives, wherein the one or more tezepelumab derivatives are high molecular weight (HMW) species, and wherein the amount of the HMW species in the composition is less than about 20%, wherein tezepelumab comprises (A) a light chain variable domain comprising: (i) a light chain CDR1 amino acid sequence set out in SEQ ID NO:3; (ii) a light chain CDR2 amino acid sequence set out in SEQ ID NO: 4; and (iii) a light chain CDR3 amino acid sequence set out in SEQ ID NO:5; and (B) a heavy chain variable domain comprising: (i) a heavy chain CDR1 amino acid sequence set out in SEQ ID NO:6; (ii) a heavy chain CDR2 amino acid sequence set out in SEQ ID NO:7 and (iii) a heavy chain CDR3 amino acid sequence set out in SEQ ID NO:8.

In various embodiments, the HMW species in the composition is less than about 20%, 15%, 10%, 5%, 4%, 3%, 2% or 1%. In various embodiments, the amount of the HMW species in the composition is from about 0.5% to about 13%, about 1% to about 11%, about 2% to about 10%, or about 3% to about 8% or about 4% to about 7%. In various embodiments, the amount of the HMW species in the composition is about 1.7% or less. In various embodiments, the amount of the HMW species in the composition is about 1.4% or less. In various embodiments, the HMW species comprises a dimer of tezepelumab.

In various embodiments, the amount of the HMW species in the composition is determined by size exclusion-high performance liquid chromatography (SE-HPLC), Sedimentation velocity ultracentrifugation (SV-AUC), or reduced sodium dodecyl sulfate capillary electrophoresis (rCE-SDS). In various embodiments, the SE-HPLC is SE-ultra HPLC (SE-UHPLC) or SE-HPLC with static light scattering (SE-HPLC-SLS). In various embodiments, the SE-HPLC is SE-UHPLC. In various embodiments, when the SE-HPLC is the SE-UHPLC, the proteins are separated isocratically using a mobile phase comprising 100 mM sodium phosphate, 250 mM sodium chloride at pH 6.8. In various embodiments, the tezepelumab and tezepelumab derivatives have a greater potency and/or tolerability than a composition comprising greater than 20% of the HWM species, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 20% of the HWM species, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

In various embodiments of the composition, (a) the tezepelumab and tezepelumab derivatives have a greater potency and/or tolerability than a composition comprising greater than 40% of the glycosylation derivatives, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR; or (b) the tezepelumab and tezepelumab derivatives comprise no more than 15% high mannose, and have less clearance than a composition having greater than 15% high mannose.

In various embodiments of the composition, (a) the tezepelumab and tezepelumab derivatives have a greater potency and/or tolerability than a composition comprising greater than 40% of the glycosylation derivatives, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR; or (b) the tezepelumab and tezepelumab derivatives comprise no more than 25% high mannose, and have less clearance than a composition having greater than 25% high mannose.

Also provided is a composition comprising tezepelumab and one or more tezepelumab derivatives, wherein the one or more tezepelumab derivatives comprise a tezepelumab fragment, and wherein the amount of the tezepelumab fragment in the composition is less than about 15%, wherein tezepelumab comprises (A) a light chain variable domain comprising: (i) a light chain CDR1 amino acid sequence set out in SEQ ID NO:3; (ii) a light chain CDR2 amino acid sequence set out in SEQ ID NO: 4; and (iii) a light chain CDR3 amino acid sequence set out in SEQ ID NO:5; and (B) a heavy chain variable domain comprising: (i) a heavy chain CDR1 amino acid sequence set out in SEQ ID NO:6; (ii) a heavy chain CDR2 amino acid sequence set out in SEQ ID NO:7 and (iii) a heavy chain CDR3 amino acid sequence set out in SEQ ID NO:8.

In various embodiments, the amount of fragments in the composition is less than about 15%, 10%, 5%, 4%, 3%, 2% or 1%. In various embodiments, the amount of the fragments in the composition is from about 0.5% to about 13%, about 1% to about 11%, about 2% to about 10%, or about 3% to about 8% or about 4% to about 7%. In various embodiments, the tezepelumab fragments are low molecular weight (LMW) or middle molecular weight (MMW) species, or combinations thereof. In various embodiments, the fragments are low molecular weight species of less than about 25 kD. In various embodiments, the fragments are middle molecular weight species having a molecular weight between about 25 to 50 kD. In various embodiments, the amount of tezepelumab fragment in the composition is determined by reduced capillary electrophoresis with sodium dodecyl sulfate (rCE-SDS). In various embodiments, the tezepelumab and tezepelumab derivatives have a greater potency and/or tolerability than a composition comprising greater than 20% of the tezepelumab fragments, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 15% of the tezepelumab fragments, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

Provided herein is a composition comprising tezepelumab and one or more tezepelumab derivatives, wherein the tezepelumab derivatives comprise isomerization derivatives, deamidation derivatives, oxidation derivatives, glycosylation derivatives, HMW species, fragments, disulfide isomers, or combinations thereof, wherein the composition has one or more of the following characteristics: (a) the amount of isomerization derivatives in the composition is about 30% or less as measured by reduced peptide mapping; (b) the amount of deamidation derivatives in the composition is about 15% or less as measured by peptide mapping; (c) the amount of oxidation derivatives in the composition is about 7% or less as measured by reduced peptide mapping; (d) the amount of glycosylation derivatives in the composition is about 75% or less as measured by glycan mapping; (e) the amount of disulfide isomers in the composition is about 40% or less as measured by non-reduced reversed phase high performance liquid chromatography (RP-HPLC); (f) the amount of HMW species in the composition is about 20% or less as measured by SE-HPLC; and/or (g) the amount of fragments in the composition is about 20% or less as measured by rCE-SDS.

In various embodiments, the tezepelumab comprises a heavy chain amino acid sequence set out in SEQ ID NO: 10 and a light chain amino acid sequence set out in SEQ ID NO: 12.

In another aspect, the disclosure provides a method for assessing the quality of a tezepelumab composition, comprising: obtaining a tezepelumab composition that contains tezepelumab and one or more tezepelumab derivatives; measuring the amount of one or more tezepelumab derivatives in the composition, wherein the tezepelumab derivatives comprise isomerization derivatives, deamidation derivatives, oxidation derivatives, glycosylation v, disulfide isoform derivatives, HMW species, fragments, or combinations thereof; comparing the measured amount of the one or more tezepelumab derivatives to a pre-determined reference criterion; and preparing a pharmaceutical formulation or pharmaceutical product of the tezepelumab composition if the comparison indicates that the pre-determined reference criterion is met. In various embodiments, the amount of isomerization derivatives is measured and the pre-determined reference criterion is about 30% or less. In various embodiments, the amount of isomerization in the tezepelumab composition is measured by reduced peptide mapping. In various embodiments, the amount of deamidation derivatives is measured and the pre-determined reference criterion is about 15% or less. In various embodiments, the amount of deamidation in the tezepelumab composition is measured by reduced peptide mapping. In various embodiments, the amount of oxidation derivatives is measured and the pre-determined reference criterion is about 7% or less. In various embodiments, the amount of oxidation in the tezepelumab composition is measured by reduced peptide mapping. In various embodiments, the amount of glycosylation derivatives is measured and the pre-determined reference criterion is about 40% or less. In various embodiments, the amount of glycosylation in the tezepelumab composition is measured by glycan mapping. In various embodiments, the amount of disulfide isoform derivatives is measured and the pre-determined reference criterion is about 75% or less. In various embodiments, the amount of disulfide isoform in the tezepelumab composition is measured by non-reduced reversed phase high performance liquid chromatography (RP-HPLC). In various embodiments, the amount of HMW species is measured and the pre-determined reference criterion is about 20% or less. In various embodiments, the amount of HMW species is measured by SE-HPLC. In various embodiments, the amount of fragments is measured and the pre-determined reference criterion is about 15% or less. In various embodiments, the amount of fragments in the tezepelumab composition is measured by rCE-SDS.

In various embodiments, the tezepelumab composition is obtained from a Chinese Hamster Ovary (CHO) cell line that expresses a nucleic acid encoding a heavy chain of SEQ ID NO: 10 and a nucleic acid encoding a light chain of SEQ ID NO: 12.

In various embodiments, the immunoglobulin, antigen binding protein or antibody is a human antibody. In various embodiments, the antibody is an IgG2 antibody. In various embodiments, the tezepelumab or derivative thereof specifically binds to a TSLP polypeptide as set forth in amino acids 29-159 of SEQ ID NO: 2. In various embodiments, both binding sites of tezepelumab or derivative thereof have identical binding to TSLP.

In various embodiments, the tezepelumab or derivative thereof binds TSLP with an affinity that is numerically no more than 10⁻⁸ M Kd.

Further contemplated is a composition comprising tezepelumab or derivatives thereof as described herein and a pharmaceutically acceptable carrier, excipient or diluent.

The disclosure also provides an isolated nucleic acid comprising a polynucleotide sequence encoding the light chain variable domain, the heavy chain variable domain, or both, of the tezepelumab or derivative thereof described herein.

The disclosure further contemplates a recombinant expression vector comprising the nucleic acid encoding tezepelumab as described herein. Also provided is a host cell comprising the expression vector.

Further contemplated herein is a method of producing a composition comprising tezepelumab or derivatives thereof that specifically binds to a TSLP polypeptide comprising amino acids 29-159 of SEQ ID NO: 2, comprising incubating the host cell under conditions that allow it to express the immunoglobulin, antigen binding protein, or antibody, wherein said host cell comprises (i) a recombinant expression vector encoding the light chain variable domain of the antigen binding protein of as described herein and a recombinant expression vector encoding the heavy chain variable domain of the antigen binding protein as described herein, or (ii) a recombinant expression vector encoding both the light chain variable domain and the heavy chain variable domain of tezepelumab.

Also provided herein is a method for treating an inflammatory disease in a subject comprising administering to the subject a therapeutically effective amount of a composition comprising tezepelumab and derivatives thereof. In various embodiments, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF). In various embodiments, the asthma is mild, moderate or severe asthma. In various embodiments, the asthma is severe asthma. In various embodiments, the asthma is eosinophilic or non-eosinophilic asthma.

In various embodiments, the method comprises administering the composition at an interval of every 2 weeks or every 4 weeks. In various embodiments, the composition is administered for a period of at least 4 months, 6 months, 9 months, 1 year or more.

In various embodiments, the antibody is an IgG2 antibody. In various embodiments, the tezepelumab or tezepelumab derivatives comprise a heavy chain variable region set out in SEQ ID NO: 10 and a light chain variable region set out in SEQ ID NO: 12, and comprises one or more of the attributes described herein.

The disclosure also provides a composition comprising tezepelumab and derivatives thereof as described herein for use in treating an inflammatory disease. In certain embodiments, the disclosure provides use of a composition comprising tezepelumab and derivatives thereof as described herein in the preparation of a medicament for treating an inflammatory disease.

Syringes, e.g., single use or pre-filled syringes, sterile sealed containers, e.g. vials, bottle, vessel, and/or kits or packages comprising any of the foregoing antibodies or compositions, optionally with suitable instructions for use, are also contemplated. In various embodiments, the administration is via pre-filled syringe or autoinjector. In various embodiments, the auto-injector is an Ypsomed YpsoMate® device.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawings and/or claims.

### BRIEF DESCRIPTION OF THE DRAWINGS

FIGS. 1A-1F are a series of leverage plots depicting relationships between potency and total CDR IsoAsp (FIGS. 1A and 1D), HMW species (FIGS. 1B and 1E), and total CDR Trp oxidation (FIGS. 1C and 1F).

### DETAILED DESCRIPTION

The structure of tezepelumab was elucidated from a variety of biological, biochemical, and biophysical techniques to provide an understanding of its structure and functional properties and assessment of critical quality attributes.

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “inflammatory disease” refers to a medical condition involving abnormal inflammation caused by the immune system attacking the body's own cells or tissues, which may result in chronic pain, redness, swelling, stiffness, and damage to normal tissues. Inflammatory diseases include, for example, asthma, chronic peptic ulcer, tuberculosis, periodontitis, sinusitis, active hepatitis, ankylosing spondylitis, rheumatoid arthritis, chronic obstructive pulmonary disease (COPD), Crohn's disease, ulcerative colitis, osteoarthritis, atherosclerosis, systemic lupus erythematosus, atopic dermatitis, eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps, idiopathic pulmonary fibrosis (IPF), and the like. In exemplary aspects, the inflammatory disease is asthma, atopic dermatitis, or COPD. In exemplary aspects, the inflammatory is asthma and, in some instances, the asthma is severe asthma, eosinophilic asthma, non-eosinophilic asthma, or low eosinophil asthma.

The term “asthma” as used herein refers to allergic, non-allergic, eosinophilic, and non-eosinophillic asthma.

The term “allergic asthma” as used herein refers to asthma that is triggered by one or more inhaled allergens. Such patients have a positive IgE fluorescence enzyme immunoassay (FEIA) level to one or more allergens that trigger an asthmatic response. Typically, most allergic asthma is associated with Th2-type inflammation.

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

The term “eosinophilic asthma” as used herein refers to an asthma patient having a screening blood eosinophil count of less than or equal to 300 cells/μL, or less than or equal to 250 cells/μL “Low eosinophilic” asthma refers to asthma patients having less than 250 cells/μL blood or serum. Alternatively, “low eosinophilic” asthma refers to asthma patients having less than 300 cells/μL blood or serum.

A “T helper (Th) 1 cytokine” or “Th1-specific cytokine” refers to cytokines that are expressed (intracellularly and/or secreted) by Th1 T cells, and include IFN-g, TNF-a, and IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-a, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds an target antigen with greater affinity than other antigens of similar sequence. It is contemplated herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-a) and the like. In various embodiments, the antibody specifically binds the target antigen, but can cross-react with an ortholog of a closely related species, e.g. an antibody may bind human protein and also bind a closely related primate protein. In various embodiments, the immunoglobulin, antigen binding protein or fragment thereof, or antibody or fragment thereof specific for TLSP binds with a Kd that is numerically less than or equal to 10⁻⁸ M. In various embodiments, an anti-TSLP antibody described herein binds at least with an affinity (Kd) of 10⁻⁸ M, 10⁻⁹ M, 10⁻¹⁰ M, 10⁻¹¹ M, 10⁻¹² M, 10⁻¹³ M or less.

The term “antibody” refers to a tetrameric glycoprotein that consists of two heavy chains and two light chains, each comprising a variable region and a constant region. “Heavy Chains” and “Light Chains” refer to substantially full length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies.

Antigen binding proteins include antibodies, antibody fragments and antibody-like proteins that can have structural changes to structure of canonical tetrameric antibodies. Antibody “variants” refer to antigen binding proteins or fragments thereof that can have structural changes in antibody sequence or function compared to a parent antibody having a known sequence. Antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, CDR-grafted antibody binding regions, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Antibody derivative” as used herein refers to antibodies, antigen binding proteins or fragments thereof comprising one or more attributes described herein, which may be characterized in terms of its chemical identity, chemical modification or structural attribute type (e.g., HMW species, fragment or isoform) and exhibits the desired biological activity.

“Valency” refers to the number of antigen binding sites on each antibody or antibody fragment that targets an epitope. A typical full length IgG molecule, or F(ab)2 is “bivalent” in that it has two identical target binding sites. A “monovalent” antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity”includes inhibiting any one or more of the following: binding of TSLP to its receptor; proliferation, activation, or differentiation of cells expressing TSLPR in the presence of TSLP; inhibition of Th2 cytokine production in a polarization assay in the presence of TSLP; dendritic cell activation or maturation in the presence of TSLP; and mast cell cytokine release in the presence of TSLP. See, e.g., U.S. Pat. No. 7,982,016 B2, column 6 and Example 8 and US 2012/0020988 A1, Examples 7-10.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

### TSLP

Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+progenitor cells (Swedin et al. Pharmacol Ther 2017; 169:13-34). TSLP signals through a heterodimeric receptor consisting of the interleukin (IL)-7 receptor alpha (IL-7Ra) chain and a common y chain-like receptor (TSLPR) (Pandey, Nat Immunol. 1:59-64, 2000; Park, J Exp Med. 192:659-669, 2000).

Human TSLP mRNA (Brightling et al., J Allergy Clin Immunol 2008; 121:5-10; quiz 1-2;Ortega et al. N Engl J Med 2014; 371:1198-207) and protein levels (Ortega et al., supra) are increased in the airways of asthmatic individuals compared to controls, and the magnitude of this expression correlates with disease severity (Brightling et al., supra). Recent studies have demonstrated association of a single nucleotide polymorphism in the human TSLP locus with protection from asthma, atopic asthma and airway hyperresponsiveness, suggesting that differential regulation of TSLP gene expression might influence disease susceptibility (Ortega et al. N Engl J Med 2014; 371:1198-207; To et al. BMC Public Health 2012; 12:204). These data suggest that targeting TSLP may inhibit multiple biological pathways involved in asthma.

Earlier non-clinical studies of TSLP suggested that after TSLP is released from airway epithelial cells or stromal cells, it activates mast cells, dendritic cells, and T cells to release Th2 cytokines (e.g., IL-4/13/5). Recently published human data demonstrated a good correlation between tissue TSLP gene and protein expression, a Th2 gene signature score, and tissue eosinophils in severe asthma. Therefore, an anti-TSLP target therapy may be effective in asthmatic patients with Th2-type inflammation (Shikotra et al, J Allergy Clin Immunol. 129(1):104-11, 2012).

Data from other studies suggest that TSLP may promote airway inflammation through Th2 independent pathways such as the crosstalk between airway smooth muscle and mast cells (Allakhverdi et al., J Allergy Clin Immunol. 123(4):958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al., Clin Exp Allergy. 39(1):89-100, 2009). These data and other emerging evidence suggest that blocking TSLP may serve to suppress multiple biologic pathways including but not limited to those involving Th2 cytokines (IL-4/13/5).

### Antibodies

It is contemplated that antibodies or antibody derivatives or antigen binding proteins specific for TSLP are useful in the treatment of inflammatory diseases, including asthma, such as severe asthma, eosinophilic asthma, no-eosinophilic/low-eosinophilic and other forms of asthma described herein, atopic dermatitis, EoE, and COPD.

Specific binding agents such as antibodies and antibody derivatives or fragments that bind to their target antigen, e.g., TSLP, are useful in the methods and compositions of the disclosure. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the invention are human antibody variants and derivatives (including antibody fragments) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies, antibody fragments, and/or antibody variants and derivatives are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein. Further examples of mammalian cells include immortalized cell lines available from the American Type Culture Collection (Manassas, VA), including, in addition to Chinese Hamster Ovary (CHO) cells, HeLa cells, baby hamster kidney (BHK) cells, monkey kidney cells (COS), human hepatocellular carcinoma cells (e.g., Hep G2), and human epithelial kidney 293 cells. Furthermore, cell lines or host systems can be chosen to ensure correct modification and processing of the tezepelumab or tezepelumab derivatives. Eukaryotic host cells that possess the cellular machinery for proper processing of the primary transcript, glycosylation, and phosphorylation of the gene product can be used. These include CHO, VERY, BHK, Hela, COS, MDCK, 293, 3T3, W138, BT483, Hs578T, HTB2, BT20 and T47D, NS0 (a murine myeloma cell line that does not endogenously produce any functional immunoglobulin chains), SP20, CRL7030 and HsS78Bst cells. Human cell lines developed by immortalizing human lymphocytes can also be used. The human cell line PER.C6® (Janssen; Titusville, NJ) can also be used to recombinantly produce monoclonal antibodies.

By way of example, tezepelumab and tezepelumab derivatives having molecular attributes as described herein may be obtained by selecting a cell clone that expresses the tezepelumab or a tezepelumab derivative having the molecular attributes. Recombinant DNA methods may be used for producing such tezepelumab or tezepelumab derivatives. For example, DNA encoding the heavy chain and light chain of the tezepelumab or tezepelumab derivatives can be inserted into a suitable expression vector (or vectors, for example one vector for the heavy chain and one for the light chain), which can be transfected into a suitable host cell, such as a cell of a mammalian cell line. Suitable expression vectors are known in the art, containing, for example, a polynucleotide that encodes the tezepelumab polypeptide linked to a promoter. The expression vector may be transferred to a host cell by conventional techniques, and the transfected cells may be cultured to produce the antibodies. Optionally, the host cells may be engineered to modulate molecular attributes. For example, to modulate fucosylation, glycosylation-competent cells may be genetically modified to alter the activity of a fucosyl-transferase or a Golgi GDP-fucose transporter. By way of example, cell line engineering to modulate glycosylation is described in PCT Pub. No. WO 2015/116315.

Clones producing the tezepelumab or tezepelumab derivatives comprising the relevant molecular attributes may be selected. By way of example, established microtiter plate-based method of clone generation and growth may be performed. Hundreds of pooled, heterogeneous cells may be sorted into single-cell cultures through processes such as fluorescence-activated cell sorting (FACS) or limiting dilution. After being allowed to recover to healthy and stable populations, these clonally-derived cells may be analyzed, and select populations are chosen for further analysis. For further analysis, clone cells may be cultured in small containers, such as spin tubes, 24-well plates, or 96-deep well plates are cultured in a “small-scale cell culture” (e.g., a 10-day fed batch process). In this small-scale process, boluses of nutrients are added periodically, and different measurements of cell growth and viability are obtained. Hundreds or even thousands of these small-scale cultures may be in parallel. At the end of the culture (e.g., the tenth day), the cells are harvested for assays and analysis. Optionally, the microtiter plate-based method of clone generation and growth (e.g., subcloning) may be substituted with the use of an automated or partially automated high-throughput and high-content screening tool, such as the Berkeley Lights Beacon™ opto-electronic cell line generation and analysis system, for example. Optionally, high throughput screening methods and machine learning tools may be used to expedite the selection of clones producing the relevant molecular attributes (See, e.g., PCT Pub. No. WO 2020/223422).

Anti-TSLP antibody tezepelumab is described in U.S. Pat. No. 7,982,016 and U.S. patent application Ser. No. 15/951,602.

Anti-TSLP antigen binding protein (including fragments thereof) useful in the present methods comprise an anti-TSLP antibody comprising a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and, b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody or antibody derivative specifically binds to a TSLP polypeptide as set forth in amino acids 29-159 of SEQ ID NO:2.

Also contemplated is an antibody or antibody derivative comprising a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and, b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody or antibody derivative specifically binds to a TSLP polypeptide as set forth in amino acids 29-159 of SEQ ID NO:2.

Tezepelumab is an exemplary anti-TSLP antibody having: a. i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8.

Tezepelumab also comprises a light chain variable domain having the amino acid sequence set out in SEQ ID NO:12; encoded by a polynucleotide sequence set out in SEQ ID NO:11; and a heavy chain variable domain having the amino acid sequence set out in SEQ ID NO:10, encoded by a polynucleotide sequence set out in SEQ ID NO:9.

In various embodiments, the anti-TSLP antibody or antibody derivative thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In various embodiments, the anti-TSLP antibody derivative is selected from the group consisting of a diabody, a triabody, a tetrabody, a Fab fragment, single domain antibody, scFv, wherein the dose is adjusted such that the binding sites to be equimolar to the those dosed by bivalent antibodies.

It is contemplated that the antibody or antibody derivative is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production. Tezepelumab is an IgG2 antibody. The sequence of the full length heavy chain and light chain of tezepelumab, including the IgG2 chain, is set out in SEQ ID NOs: 13 and 14, respectively.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinity affinities, (4) inhibit formation of high molecular weight (HMW) species, and/or (5) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Methods of Making

Tezepelumab compositions of the disclosure can be prepared by recombinantly expressing nucleic acids encoding the heavy chain and light chain in a host cell, partially purifying or purifying tezepelumab from host cell cultures or host cell lysates, and analyzing the resulting compositions for one or more of the tezepelumab derivatives detailed herein according to the methods described in more detail below.

For recombinant production of tezepelumab or tezepelumab derivatives, one or more nucleic acids encoding the heavy chain (e.g. heavy chain polypeptide comprising the amino acid sequence of SEQ ID NO: 10) and light chain (e.g. light chain polypeptide comprising the amino acid sequence of SEQ ID NO: 12) is inserted into one or more expression vectors. The nucleic acid encoding the heavy chain and the nucleic acid encoding the light chain can be inserted into a single expression vector or they can be inserted into separate expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Vectors may also include one or more selectable marker genes to facilitate selection of host cells into which the vectors have been introduced. Exemplary nucleic acids encoding the heavy and light chains of tezepelumab as well as suitable signal peptide sequences and other components for expression vectors for recombinantly expressing tezepelumab are described in U.S. Pat. No. 7,982,016, which is hereby incorporated by reference in its entirety, and set out in SEQ ID NO: 9 and SEQ ID NO: 11 herein.

After the expression vector has been constructed and the one or more nucleic acid molecules encoding the heavy and light chain components of tezepelumab or derivative thereof has been inserted into the proper site(s) of the vector or vectors, the completed vector(s) may be inserted into a suitable host cell for amplification and/or polypeptide expression. The transformation of an expression vector for tezepelumab or derivative thereof into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989).

A host cell, when cultured under appropriate conditions, synthesizes tezepelumab or derivative thereof that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antibodies from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. CHO cells are preferred host cells in some embodiments for expressing tezepelumab or derivatives thereof.

Host cells are transformed or transfected with the above-described expression vectors for production of tezepelumab or derivatives thereof and are cultured in conventional nutrient media modified as appropriate for inducing promoters, selecting transformants, or amplifying the genes encoding the desired sequences. The host cells used to produce tezepelumab or derivatives thereof may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinary skilled artisan.

Upon culturing the host cells, the antibody can be produced intracellularly, in the periplasmic space, or directly secreted into the medium. If the antibody is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the antibody is secreted into the culture medium, the antibody can be separated from host cells through centrifugation or microfiltration, and optionally, subsequently concentrated through ultrafiltration. Tezepelumab or derivatives thereof can be further purified or partially purified using, for example, one or more chromatography steps, such as affinity chromatography (e.g. protein A or protein G affinity chromatography), cation exchange chromatography, anion exchange chromatography, hydroxyapatite chromatography, hydrophobic interaction chromatography, or mixed mode chromatography.

Once a tezepelumab composition is produced or obtained, the composition may be evaluated for the presence and amount of one or more tezepelumab derivatives described herein, including isomerization derivatives (including isomerization intermediates thereof), deamidation derivatives (including deamidation intermediates thereof), oxidation derivatives, glycosylation derivatives, disulfide isoform derivatives and size derivatives (e.g. HMW species or fragments). Accordingly, the present disclosure includes methods for assessing the quality of a tezepelumab composition, comprising obtaining a tezepelumab composition that contains tezepelumab and one or more tezepelumab derivatives; measuring the amount of one or more tezepelumab derivatives in the composition; comparing the measured amount of the one or more tezepelumab derivatives to a pre-determined reference criterion; and preparing a pharmaceutical formulation or pharmaceutical product of the tezepelumab composition if the comparison indicates that the pre-determined reference criterion is met. In some embodiments, the methods comprise one, two, three, four, five, six or seven of: (1) measuring the amount of isomerization derivatives (including isomerization intermediates thereof) in the composition, (2) measuring the amount of deamidation derivatives (including deamidation intermediates thereof) in the composition, (3) measuring the amount of oxidation derivatives in the composition, (4) measuring the amount of glycosylation derivatives in the composition, (5) measuring the amount of disulfide isoform derivatives in the composition, (6) measuring the amount of HMW species in the composition, and/or (7) measuring the amount of fragments in the composition. In certain embodiments, all seven measurements are performed on a tezepelumab composition.

The pre-determined reference criterion for each tezepelumab derivative can be a threshold amount or range of amounts of the derivative that do not significantly affect the potency and/or tolerability of the tezepelumab composition, e.g., for safety purposes during administration or for inhibiting ligand-induced activation of the TSLP receptor. For instance, the pre-determined reference criterion for each tezepelumab derivative can be any of the limits or ranges disclosed herein for each of the derivatives as tezepelumab compositions with these limits/ranges of the derivatives had comparable potency and/or tolerability to tezepelumab compositions evaluated in clinical trials and shown to have clinical efficacy. It will be appreciated that pre-determined reference criteria described herein may be specified prior to the commencement of a method as described herein.

In certain embodiments of the methods, if the measured amount of the tezepelumab derivative in the composition meets the pre-determined reference criterion, then the tezepelumab composition can be classified as acceptable and progressed to the next step in the manufacturing or distribution process, such as for example, by preparing a pharmaceutical formulation of the composition (e.g. by combining with one or more excipients or diluents); by preparing a pharmaceutical product of the composition (e.g., by filling into vials, syringes, autoinjectors, or other containers or delivery devices); packaging the composition with instructions for use, diluents, and/or delivery devices; or releasing the composition for commercial sale or shipping to distributors. In some embodiments of the methods, a pharmaceutical formulation of the tezepelumab composition is prepared if the measured amount of the tezepelumab derivative in the composition meets the pre-determined reference criterion. In other embodiments of the methods, a pharmaceutical product of the tezepelumab composition is prepared if the measured amount of the tezepelumab derivative in the composition meets the pre-determined reference criterion. Methods of preparing pharmaceutical formulations and pharmaceutical products of tezepelumab compositions are described in more detail below. If the measured amount of the tezepelumab derivative in the composition does not meet the pre-determined reference criterion, then, in some embodiments of the methods, the tezepelumab composition can be classified as unacceptable and discarded, destroyed, or subject to additional manufacturing steps, such as additional purification to remove or reduce the amount of the tezepelumab derivative in the composition such that the pre-determined reference criterion is met.

In one embodiment, the methods for assessing the quality of a tezepelumab composition comprise obtaining a tezepelumab composition that contains tezepelumab and tezepelumab isomerization derivatives (including isomerization intermediates thereof); measuring the amount of the isomerization derivatives in the composition; comparing the measured amount of the isomerization derivatives to a pre-determined reference criterion; and preparing a pharmaceutical formulation or pharmaceutical product of the tezepelumab composition if the comparison indicates that the pre-determined reference criterion is met. The pre-determined reference criterion for the amount of isomerization derivatives in a tezepelumab composition can be less than about 30%, for example less than about 25%, about 20% or less, about 17% or less, about 15% or less, about 12% or less, about 10% or less, about 8% or less, about 6% or less, or about 4% or less. In one embodiment, the pre-determined reference criterion for the amount of isomerization derivatives in a tezepelumab composition is about 15% or less. In another embodiment, the pre-determined reference criterion for the amount of isomerization derivatives in a tezepelumab composition is about 13% or less. In another embodiment, the pre-determined reference criterion for the amount of isomerization derivatives in a tezepelumab composition is less than about 10%, about 8%, about 5%, about 3% or about 2%. In some embodiments, the pre-determined reference criterion for the amount of isomerization derivatives in a tezepelumab composition can be a range of amounts, for example, from about 0.5% to about 13% of a tezepelumab composition, from about 1% to about 10% of a tezepelumab composition, or from about 0.5% to about 5% of a tezepelumab composition. In another embodiment, the pre-determined reference criterion for the amount of isomerization derivative in a tezepelumab composition is less than about 5%, 4%, 3%, 2% or 1% isomerization at D54 of SEQ ID NO: 7, in either or both variable region chains. In various embodiments, the pre-determined reference criterion for the amount of isomerization derivative in a tezepelumab composition is less than about 13%, about 10%, about 8%, about 5%, about 3% or about 2% isomerization at one or more of D49, D50 or D52 of SEQ ID NO: 4. In certain embodiments, the amount of isomerization derivatives in a tezepelumab composition is measured by reduced peptide mapping.

In one embodiment, the methods for assessing the quality of a tezepelumab composition comprise obtaining a tezepelumab composition that contains tezepelumab and tezepelumab deamidation derivatives (including deamidation intermediates thereof); measuring the amount of the deamidation derivatives in the composition; comparing the measured amount of the deamidation derivatives to a pre-determined reference criterion; and preparing a pharmaceutical formulation or pharmaceutical product of the tezepelumab composition if the comparison indicates that the pre-determined reference criterion is met. The pre-determined reference criterion for the amount of deamidation derivatives in a tezepelumab composition can be less than about 15%, for example about 13% or less, about 10% or less, about 8% or less, about 6% or less, about 4% or less, about 3% or less, about 2% or less, or about 1% or less. In one embodiment, the pre-determined reference criterion for the amount of deamidation derivatives in a tezepelumab composition is about 7% or less. In another embodiment, the pre-determined reference criterion for the amount of deamidation derivative sin a tezepelumab composition is about 5% or less. In another embodiment, the pre-determined reference criterion for the amount of deamidation derivatives in a tezepelumab composition is about 2% or less. In some embodiments, the pre-determined reference criterion for the amount of deamidation derivatives in a tezepelumab composition can be a range of amounts, for example, from about 0.4% to about 10% of a tezepelumab composition, from about 1% to about 7% of a tezepelumab composition, or from about 0.1% to about 4% of a tezepelumab composition. In various embodiments, the pre-determined reference criterion for the amount of deamidation derivatives in a tezepelumab composition is less than about 3% deamidation at N25/N26 in LCDR1 set out in SEQ ID NO: 3. In various embodiments, the pre-determined reference criterion for the amount of deamidation derivatives in a tezepelumab composition is less than about 13% deamidation at N316 in the heavy chain set out in SEQ ID NO: 13, and/or N385/390 in the heavy chain set out in SEQ ID NO: 13. In certain embodiments, the amount of deamidation derivatives in a tezepelumab composition is measured by reduced peptide mapping.

In one embodiment, the methods for assessing the quality of a tezepelumab composition comprise obtaining a tezepelumab composition that contains tezepelumab and tezepelumab oxidation derivatives; measuring the amount of the oxidation derivatives in the composition; comparing the measured amount of the oxidation derivatives to a pre-determined reference criterion; and preparing a pharmaceutical formulation or pharmaceutical product of the tezepelumab composition if the comparison indicates that the pre-determined reference criterion is met. The pre-determined reference criterion for the amount of oxidation derivatives in a tezepelumab composition can be less than about 7% or less, about 6% or less, about 4% or less, about 3% or less, about 2% or less, or about 1% or less. In one embodiment, the pre-determined reference criterion for the amount of oxidation derivatives in a tezepelumab composition is about 7% or less. In another embodiment, the pre-determined reference criterion for the amount of oxidation derivatives in an tezepelumab composition is about 5% or less. In another embodiment, the pre-determined reference criterion for the amount of oxidation derivatives in an tezepelumab composition is about 3% or less. In some embodiments, the pre-determined reference criterion for the amount of oxidation derivatives in a tezepelumab composition can be a range of amounts, for example, from about 0.1% to about 7% of a tezepelumab composition, from about 0.4% to about 5% of a tezepelumab composition, or from about 0.8% to about 3% of a tezepelumab composition. In another embodiment, the pre-determined reference criterion for the amount of oxidation derivatives in an tezepelumab composition is about 7% or less oxidation at W102 in HCDR3 set out in SEQ ID NO: 8, or about 6% or less, or about 5% or less, or about 3% or less. In certain embodiments, the amount of oxidation derivatives in a tezepelumab composition is measured by reduced peptide mapping.

In one embodiment, the methods for assessing the quality of a tezepelumab composition comprise obtaining a tezepelumab composition that contains tezepelumab and tezepelumab glycosylation derivatives; measuring the amount of the glycosylation derivatives in the composition; comparing the measured amount of the glycosylation derivatives to a pre-determined reference criterion; and preparing a pharmaceutical formulation or pharmaceutical product of the tezepelumab composition if the comparison indicates that the pre-determined reference criterion is met. The pre-determined reference criterion for the amount of glycosylation derivatives in a tezepelumab composition can be less than about 40%, for example about 35% or less, about 30% or less, about 25% or less, about 20% or less, about 17% or less, about 15% or less, about 12% or less, about 10% or less, about 8% or less, about 6% or less, or about 4% or less. In one embodiment, the pre-determined reference criterion for the amount of glycosylation derivatives in a tezepelumab composition is about 30% or less. In another embodiment, the pre-determined reference criterion for the amount of glycosylation derivatives in a tezepelumab composition is about 20% or less. In another embodiment, the pre-determined reference criterion for the amount of glycosylation derivatives in a tezepelumab composition is about 15% or less. In another embodiment, the pre-determined reference criterion for the amount of glycosylation derivatives in a tezepelumab composition is about 10% or less. In some embodiments, the pre-determined reference criterion for the amount of glycosylation derivatives in a tezepelumab composition can be a range of amounts, for example, from about 1% to about 40% of a tezepelumab composition, from about 4% to about 30% of a tezepelumab composition, from about 2% to about 20% of a tezepelumab composition, or from about 5% to about 15% of a tezepelumab composition. In various embodiments, the pre-determined reference criterion for the amount of glycosylation derivatives in a tezepelumab composition is high mannose glycosylation in the composition of about 17% or less, about 15% or less, about 12% or less, about 10% or less, about 8% or less, about 5% or less, or about 4% or less. In various embodiments, the pre-determined reference criterion for the amount of glycosylation derivatives in a tezepelumab composition is high mannose glycosylation in the composition of about 25% or less, about 23% or less (e.g., about 23.1% or less), about 21% or less, about 19% or less, about 17% or less, about 15% or less, about 12% or less, about 10% or less, about 8% or less, about 6% or less, about 5% or less, or about 4% or less. In various embodiments, the pre-determined reference criterion for the amount of glycosylation derivatives in a tezepelumab composition is high mannose glycosylation in the composition of about 23.1% or less. In various embodiments, the pre-determined reference criterion for the amount of glycosylation derivatives in a tezepelumab composition is galactosylation in the composition of about 30% or less, about 25% or less, about 20% or less, about 17% or less, about 15% or less, about 12% or less, about 10% or less, about 8% or less, about 5% or less, or about 4% or less. In various embodiments, the pre-determined reference criterion for the amount of glycosylation derivatives in a tezepelumab composition is afucosylated glycosylation of about 5% or less, about 4% or less, about 3% or less, about 2% or less, about 1% or less. In certain embodiments, the amount of glycosylation derivatives in a tezepelumab composition is measured by glycan mapping.

In one embodiment, the methods for assessing the quality of a tezepelumab composition comprise obtaining a tezepelumab composition that contains tezepelumab and tezepelumab disulfide isoform derivatives; measuring the amount of the disulfide isoform derivatives in the composition; comparing the measured amount of the disulfide isoform to a pre-determined reference criterion; and preparing a pharmaceutical formulation or pharmaceutical product of the tezepelumab composition if the comparison indicates that the pre-determined reference criterion is met. The pre-determined reference criterion for the amount of disulfide isoform derivatives in a tezepelumab composition can be less than about 75%, for example about 70% or less, about 65% or less, about 55% or less, about 50% or less, about 45% or less, about 40% or less, about 35% or less, about 30% or less, about 25% or less, about 20% or less, about 17% or less, about 15% or less, about 12% or less, about 10% or less, about 8% or less, about 6% or less, or about 4% or less. In one embodiment, the pre-determined reference criterion for the amount of disulfide isoform derivatives in a tezepelumab composition is about 50% or less. In one embodiment, the pre-determined reference criterion for the amount of disulfide isoform derivatives in a tezepelumab composition is about 35% or less. In one embodiment, the pre-determined reference criterion for the amount of disulfide isoform derivatives in a tezepelumab composition is about 25% or less. In one embodiment, the pre-determined reference criterion for the amount of disulfide isoform derivatives in a tezepelumab composition is about 15% or less. In another embodiment, the pre-determined reference criterion for the amount of disulfide isoform derivatives in a tezepelumab composition is about 10% or less. In another embodiment, the pre-determined reference criterion for the amount of disulfide isoform derivatives in a tezepelumab composition is about 8% or less. In some embodiments, the pre-determined reference criterion for the amount of disulfide isoform derivatives in a tezepelumab composition can be a range of amounts, for example, from about 10% to about 70% of a tezepelumab composition, from about 15% to about 50% of a tezepelumab composition, or from about 20% to about 40% of a tezepelumab composition. In one embodiment, the pre-determined reference criterion for the amount of disulfide isoform derivatives in a tezepelumab composition is about 50% or less IgG2-A/B isoform. In one embodiment, the pre-determined reference criterion for the amount of disulfide isoform derivatives in a tezepelumab composition is about 5% or less IgG2-B isoform. In certain embodiments, the amount of disulfide isoform derivatives in a tezepelumab composition is measured by reverse phase-HPLC.

In another embodiment, the methods for assessing the quality of a tezepelumab composition comprise obtaining a tezepelumab composition that contains tezepelumab and HMW species of tezepelumab; measuring the amount of the HMW species in the composition; comparing the measured amount of the HMW species to a pre-determined reference criterion; and preparing a pharmaceutical formulation or pharmaceutical product of the tezepelumab composition if the comparison indicates that the pre-determined reference criterion is met. The pre-determined reference criterion for the amount of HMW species in a tezepelumab composition can be less than about 20%, for example about 15% or less, about 12% or less, about 10% or less, about 9% or less, about 8% or less, about 7% or less, about 6% or less, about 5% or less or about 4% of less. The pre-determined reference criterion for the amount of HMW species in a tezepelumab composition can be less than about 3.0%, for example about 2.5% or less, about 2.4% or less, about 2.3% or less, about 2.2% or less, about 2.1% or less, about 2.0% or less, about 1.8% or less, about 1.6% or less, about 1.4% or less, about 1.2% or less, about 1.0% or less, about 0.8% or less, about 0.6% or less, or about 0.4% or less. In one embodiment, the pre-determined reference criterion for the amount of HMW species in a tezepelumab composition is about 2.5% or less. In another embodiment, the pre-determined reference criterion for the amount of HMW species in a tezepelumab composition is about 1.7% or less. In another embodiment, the pre-determined reference criterion for the amount of HMW species in a tezepelumab composition is about 1.4% or less. In yet another embodiment, the pre-determined reference criterion for the amount of HMW species in a tezepelumab composition is about 1.2% or less. In still another embodiment, the pre-determined reference criterion for the amount of HMW species in a tezepelumab composition is about 0.6% or less. The pre-determined reference criterion for the amount of HMW species in a tezepelumab composition can, in some embodiments, be a range of amounts, for example from about 0.3% to about 2.4% of a tezepelumab composition, from about 0.6% to about 2.1% of a tezepelumab composition, from about 0.4% to about 1.2% of a tezepelumab composition, or from about 0.6% to about 1.4% of a tezepelumab composition. In certain embodiments, the amount of HMW species in a tezepelumab composition is measured by SE-HPLC, e.g. by SE-UHPLC, SE-HPLC-SLS, or Sedimentation velocity ultracentrifugation (SV-AUC).

In another embodiment, the methods for assessing the quality of a tezepelumab composition comprise obtaining a tezepelumab composition that contains tezepelumab and fragments (e.g., LMW or MMW) of tezepelumab; measuring the amount of the fragments in the composition; comparing the measured amount of the fragments to a pre-determined reference criterion; and preparing a pharmaceutical formulation or pharmaceutical product of the tezepelumab composition if the comparison indicates that the pre-determined reference criterion is met. The pre-determined reference criterion for the amount of HMW species in a tezepelumab composition can be about 15% or less, about 12% or less, about 10% or less, about 9% or less, about 8% or less, about 7% or less, about 6% or less, about 5% or less or about 4% of less. The pre-determined reference criterion for the amount of fragments in a tezepelumab composition can be less than about 3.0%, for example about 2.5% or less, about 2.4% or less, about 2.3% or less, about 2.2% or less, about 2.1% or less, about 2.0% or less, about 1.8% or less, about 1.6% or less, about 1.4% or less, about 1.2% or less, about 1.0% or less, about 0.8% or less, about 0.6% or less, or about 0.4% or less. In one embodiment, the pre-determined reference criterion for the amount of fragments in a tezepelumab composition is about 2.5% or less. In another embodiment, the pre-determined reference criterion for the amount of fragments in a tezepelumab composition is about 1.7% or less. In another embodiment, the pre-determined reference criterion for the amount of fragments in a tezepelumab composition is about 1.4% or less. In yet another embodiment, the pre-determined reference criterion for the amount of fragments in a tezepelumab composition is about 1.2% or less. In still another embodiment, the pre-determined reference criterion for the amount of fragments in a tezepelumab composition is about 0.6% or less. The pre-determined reference criterion for the amount of fragments in a tezepelumab composition can, in some embodiments, be a range of amounts, for example from about 0.3% to about 2.4% of a tezepelumab composition, from about 0.6% to about 2.1% of a tezepelumab composition, from about 0.4% to about 1.2% of a tezepelumab composition, or from about 0.6% to about 1.4% of a tezepelumab composition. In certain embodiments, the amount of fragments in a tezepelumab composition is measured by rCE-SDS.

In certain embodiments of the methods of the invention, the methods comprise:
(a) obtaining a tezepelumab composition that contains tezepelumab and one or more tezepelumab derivatives, wherein the tezepelumab derivatives comprise isomerization derivatives, deamidation derivatives, oxidation derivatives, glycosylation v, disulfide isoform derivatives, HMW species, fragments or combinations thereof;
(b) evaluating the tezepelumab composition by performing one or more of the following:
   (i) measuring the amount of isomerization derivatives in the composition by reduced peptide mapping and comparing the measured amount to a pre-determined reference criterion of about 30% or less;
   (ii) measuring the amount of deamidation derivatives in the composition by reduced peptide mapping and comparing the measured amount to a pre-determined reference criterion of about 15% or less;
   (iii) measuring the amount of oxidation derivatives in the composition by reduced peptide mapping and comparing the measured amount to a pre-determined reference criterion of about 7% or less;
   (iv) measuring the amount of glycosylation derivatives in the composition by glycan mapping and comparing the measured amount to a pre-determined reference criterion of about 40% or less;
   (vi) measuring the amount of disulfide isoform derivatives in the composition non-reduced reversed phase high performance liquid chromatography (RP-HPLC) and comparing the measured amount to a pre-determined reference criterion of about 75% or less;
   (vi) measuring the amount of HMW species in the composition by the pre-peaks in SE-HPLC and comparing the measured amount to a pre-determined reference criterion of about 20% or less; and/or
   (vii) measuring the amount of fragments in the composition by the pre-peaks in rCE-SDS and comparing the measured amount to a pre-determined reference criterion of about 15% or less and
(c) preparing a pharmaceutical formulation or pharmaceutical product of the tezepelumab composition if the comparison or comparisons in step (b) indicate that the pre-determined reference criterion/criteria are met. In some embodiments, all steps (b)(i), (b)(ii), (b)(iii), (b)(iv), b)(v), (b)(vi), and b)(vii) are performed. In other embodiments, only steps (b)(vi) and (b)(vii) are performed. In certain embodiments, steps (b)(iv), (b)(vi) and (b)(vii) are performed.

### Identification of Attributes Contributing to Protein Binding

In order to determine attributes that contribute to protein binding and activity, anti-TSLP antibody tezepelumab as described herein is placed in a condition that leads to a change in its structure, for example, a change in the structure of an amino acid of the therapeutic protein, leading to the formation of a derivative of the therapeutic protein. In exemplary aspects, the changed structure of an amino acid is referred to as an “attribute” and may be characterized in terms of its chemical identity or attribute type and location within the amino acid sequence of the antigen binding protein, e.g., the position of the amino acid on which the attribute is present. For example, asparagine and glutamine residues are susceptible to deamidation. A deamidated asparagine at position 10 of a protein amino acid sequence is an example of an attribute. A list of exemplary attribute types for particular amino acids is provided in Table A. As such, a “structure” as used herein can comprise, consist essentially of, or consisting of an attribute type listed in Table A, or a combination of two or more attribute types listed in Table A. It will be understood that attributes are examples of structures, and unless stated otherwise, wherever a “structure” is mentioned herein, an attribute is contemplated as an example of the structure. For example, high molecular weight species (HMW) and fragments are also examples of attributes.

TABLE A

| Exemplary Attribute Type | Amino acid residue |
|---|---|
| deamidation | Asn, Gln |
| deamination | Glu, Ser, Gly |
| glycation, hydroxylysine | Lys |
| glycosylation | Asn |
| cyclization | N-terminal Gln, N-terminal Glu |
| oxidation | Met, Trp, His |
| isomerization | Asp |
| fragmentation/clipping | Asp/Pro |

As an immunoglobulin or fragment thereof, antibody or antigen binding protein, such as tezepelumab, comprises multiple amino acids, an antibody or antigen binding protein described herein may have more than one attribute (e.g., more than one amino acid having a changed structure) and may be described in terms of its attribute profile. As used herein, the term “attribute profile” refers to a listing of an antigen binding protein's attributes. In various instances, the attribute profile provides the chemical identity or attribute type, e.g., deamidation, optionally, relative to the native structure of the therapeutic protein. In various instances, the attribute profile provides the location of the attribute, e.g., the position of the amino acid on which the attribute is present. An attribute profile in some aspects, provides a description of all attributes present on the antigen binding protein. In other aspects, an attribute profile provides a description of a subset of attributes present on the protein. For example, an attribute profile may provide only those attributes that are present in a particular portion of the protein, e.g., the constant region, the variable region, the CDRs (such as the three light chain CDRs and the three heavy chain CDRs). A species of a therapeutic protein such as an antibody or antigen binding protein is characterized by the attribute(s) present on the protein. A species of an antibody or antigen binding protein may differ from another species of the same protein by having a different attribute profile. When two therapeutic proteins have differing attribute profiles, the therapeutic proteins represent two different species or derivatives of the therapeutic protein. When two therapeutic proteins have identical attribute profiles, the therapeutic proteins are considered as the same species or derivative of the therapeutic protein.

In various instances, the immunoglobulin, antibody or antigen binding protein is placed in a condition that leads to a change in its structure, e.g., formation of one or more attributes, and the change in structure may alter the affinity of the therapeutic protein for its target. In various aspects, the immunoglobulin, antibody or antigen binding protein is placed in a condition that leads to a change in its structure, e.g., formation of one or more attributes, and the change in structure reduces the affinity of the antigen binding protein for its target. The reduced affinity in some aspects leads to a partial or total loss of the ability of the immunoglobulin, antibody or antigen binding protein to interact with (e.g., bind to) a target. In various instances, the partial or total loss of the ability of the immunoglobulin, antibody or antigen binding protein to interact with (e.g., bind to) a target ultimately reduces the antigen binding protein's efficacy. In alternative instances, the immunoglobulin, antibody or antigen binding protein is placed in a condition that leads to a change in its structure, e.g., formation of one or more attributes, and the change in structure does not alter the affinity of the immunoglobulin, antibody or antigen binding protein for its target. In various aspects, the change in structure does not reduce the affinity of the protein for its target. Without being bound to any particular theory, the methods of the present disclosure advantageously distinguish with precision and accuracy those attributes of an immunoglobulin, antibody or antigen binding protein that affect an interaction between the immunoglobulin, antibody or antigen binding protein and the target from attributes that do not affect the interaction.

In various aspects, a composition herein comprises a population of species or derivatives of the immunoglobulin, antigen binding protein or fragment thereof, or antibody or fragment thereof. In various instances, the population is a homogenous population of the immunoglobulin, antigen binding protein or fragment thereof, or antibody or fragment thereof, optionally, each of the proteins present in the composition sample are the same species or derivative. In various instances, the population is a heterogeneous population comprising at least two different species or derivatives of the immunoglobulin, antigen binding protein or fragment thereof, or antibody or fragment thereof having an attribute described herein. In various aspects, the heterogeneous population comprises at least 2, at least 3, at least 4, at least 5, at least 6, at least 7 or more different species or derivative of the immunoglobulin, antigen binding protein or fragment thereof, or antibody or fragment thereof. Optionally, the heterogeneous population comprises more than 7, more than 8, more than 9, more than 10, more than 20, more than 30, more than 40, more than 50 different species or derivatives of the protein. Each species or derivative of the population in some aspects has a unique attribute profile. In exemplary instances, the species of the immunoglobulin, antigen binding protein or fragment thereof, or antibody or fragment thereof are the only proteins present in the composition. In some aspects, the composition comprises (i) the population immunoglobulin, antigen binding protein or fragment thereof, or antibody or fragment thereof immunoglobulin, antigen binding protein or fragment thereof, or antibody or fragment thereof and (ii) a pharmaceutically-acceptable carrier, diluent, excipient, or a combination thereof. In some embodiments, at least 80%, 85%, 90%, 95%, or 99% of immunoglobulin, antigen binding protein or fragment thereof, or antibody or fragment thereof of the heterogeneous population comprises an attribute as described herein. In some embodiments, no more than 20%, 15%, 10%, 5%, or 1% of immunoglobulin, antigen binding protein or fragment thereof, or antibody or fragment thereof of the heterogeneous population comprises an attribute as described herein.

### Separation

In exemplary embodiments, the present disclosure comprises methods for separating a mixture comprising different species of the antigen into at least two fractions. In some aspects, the mixture is separated into multiple (e.g., 2, 3, 4, 5, 6, 7, 8, 9, 10, or more) fractions.

In some aspects, the separation step of the presently disclosed methods preserves native folding, high-order structure and binding ability of the antigen binding protein and its target. In various aspects, the mixture is separated into an unbound fraction comprises unbound antibody or antigen binding proteins or targets and a bound fraction comprises antibody/antigen binding protein-target complexes.

Suitable methods and techniques for separating mixtures into fractions are known in the art. See, e.g., Coskun, North Clin Istanb 3(2): 156-160 (2016); Snyder et al., Practical HPLC Method Development, 2nd ed., John Wiley & Sons, Inc. 1997; Snyder et al., Introduction to Modern Liquid Chromatography, John Wiley & Sons, Inc., Hoboken, N J, 2010; Heftmann, Chromatography: Fundamentals and applications of chromatography and related differential migration methods, 6th ed., Volume 69A, Elsevier, Amsterdam, Netherlands, 2004; Mori and Barth, Size Exclusion Chromatography, Springer-Verlag, Berlin, 1999. In some aspects, the separation is based on charge, such as, e.g., ion exchange chromatography, capillary isoelectric focusing (clEF) and/or capillary zone electrophoresis (CZE) or is based on hydrophobicity, such as, e.g., separation in reverse phase (RP; e.g., RP-HPLC) and hydrophobic interaction chromatography (HIC-HPLC). In various aspects, the separation is based on size such as, e.g., size exclusion chromatography (SEC; e.g., SE-HPLC), sodium dodecyl sulfate-polyacrylamide gel electrophoresis (SDS-PAGE), capillary electrophoresis with sodium dodecyl sulfate (CE-SDS). The methods described herein are used for detecting product oxidation of Met or Trp residues, fragmentation/clipping, isomerization of Asp, deamidation, formation of pyroglutamic acid at the N-terminus. In various embodiments, the mixture is separated into at least two fractions using a technique that separates components of a mixture based on size, charge, hydrophobicity, affinity for a capture molecule, or a combination thereof. In various instances, the technique is size exclusion chromatography (SEC), affinity chromatography, precipitation using beads or cells, free flow fractionation (FFF), ion exchange chromatography (IEX), cation exchange chromatography (CEX), hydrophobic interaction chromatography (HIC), or ultracentrifugation (UC). Optionally, the mixture is separated into at least two fractions using a technique that separates components of a mixture based on size, optionally, wherein the technique is size exclusion chromatography (SEC).

In various aspects, the mixture is separated into at least two fractions using a technique that separates components of a mixture based on affinity for a capture molecule bound to a solid support, optionally, a bead or a cell. In various instances, the mixture is separated by (i) adding the mixture to a container, e.g., a tube, comprising beads bound to the capture molecule or cells expressing at its surface the capture molecule, (ii) centrifuging the container (e.g., tube) to obtain a supernatant and a pellet, (iii) collecting the supernatant from the pellet to obtain the unbound fraction, (iv) releasing the bound fraction from the pellet with a solution, (v) centrifuging the container (e.g., tube) comprising the pellet and the solution to obtain a second supernatant comprising the bound fraction and a second pellet comprising the beads or cells, and (vi) collecting the second supernatant to obtain the bound fraction. The mixture in some aspects is separated by (i) adding the mixture to a column comprising beads bound to the capture molecule to obtain a flow-through and a bound fraction (ii) collecting the flow-through to obtain the unbound fraction, (iii) releasing the bound fraction from the beads with a solution and collecting the solution comprising the bound fraction. Suitable solid supports include, for example, beads, resin, paper, optionally, made of cellulose, silica, alumina, glass, plastic, or a combination thereof. In exemplary aspects the capture molecule bound to the solid support is a protein. The capture molecule may be identical to the target. Advantageously, the capture molecule is not limited to any particular molecule.

In various embodiments of the method of identifying attributes of an immunogolobuiln, antigen binding protein (e.g., tezepelumab) or target that affect an interaction between the antigen binding protein and the target, for each of the unbound fraction and bound fraction, the method comprises identifying and quantifying the abundance of each attribute present on a species or derivative of the antigen binding protein or target, wherein, when the abundance of an attribute in the unbound fraction is greater than the abundance of the attribute in the bound fraction, the attribute negatively affects the interaction between the antigen binding protein and the target. In various aspects, the method comprises using a mass spectrometer to identify and quantify the abundance of each attribute of the species of the antigen binding protein or target in each of the unbound fraction and bound fraction.

In various embodiments of the method of determining an effect of a known attribute present on a species of an antigen binding protein or target on an interaction between the antigen binding protein and the target, the method comprises for each of the unbound fraction and bound fraction, quantifying the abundance of the known attribute, wherein, when the abundance of the known attribute in the unbound fraction is greater than the abundance of the known attribute in the bound fraction, the known attribute has a negative effect on the interaction between the antigen binding protein and the target. In various aspects, the method comprises using a mass spectrometer to quantify the abundance of the known attribute in each of the unbound fraction and bound fraction.

Stability refers to resistance to chemical modifications of amino acid residues and biophysical protein modifications, such as formation of HMW species during stress conditions which may occur during manufacturing, storage and/or additional or alternative stress conditions. For methods and immunoglobulins, antigen binding proteins, and fragments thereof of embodiments described herein, “stability” and/or “HMW” species, may be determined using size exclusion chromatography (SEC). A composition comprising the immunoglobulin, antigen binding protein, or fragment or derivative may be separated by SEC, such as SEC-UV. The SEC may use a mobile phase comprising 100 mM sodium phosphate and 250 mM NaCl (pH 6.8), the flow rate may be set at 0.5 ml/min, the column temperature may be set at 37° C., the run time may be 35 minutes, and the auto sampler may be set at 4° C. An example of a suitable column for SEC includes a gel column comprising silica particles comprising a diol functional group and having a mean diameter of 5 μm and a mean pore size of about 25 nM (available commercially, for example, as a G3000SWxl column from TOSOH Bioscience). For SEC-UV, ultraviolet/Visible spectrometry (UV/VIS) detection may be performed at 214 nm and 280 nm. It will be appreciated that following separation, peaks representing the monomer and HMW species can elute at different times in the SEC elution profile.

Following SEC analysis, peptide mapping may optionally be performed, and peptide modifications associated with bound and unbound species may be identified, for example as described herein and/or in International Pub. No. WO 2020/247790. For peptide mapping, the eluting fractions may be collected using a filter with a molecular weight cut-off (for example, greater than 10 kDa) and eluted with a 7.5 M guanidine elution buffer. To determine chemical modifications affecting binding to antigen, stressed immunoglobulin (or antigen binding protein or fragment thereof) and antigen may be mixed together and separated on earlier eluting antigen-bound complex and later eluting unbound immunoglobulin (or antigen binding protein or fragment thereof). To determine chemical modifications impacting or correlating with HMW, monomeric and HMW species may be collected.

It will be appreciated that “affinity” or “binding” may be determined by surface plasmon resonance (SPR), bio-layer interferometry, or also by SEC binding affinity experiments as described herein. Unless stated otherwise herein or necessitated otherwise by scientific context, “affinity” will be understood to refer to affinity as measured by SPR. Kd value may be measured by SPR using a biosensor system such as a BIAcore@ system. The analysis with the BIAcore@ system may comprise analyzing the binding and dissociation of an antigen (e.g., TSLP) from chips with immobilized molecules (e.g., anti-TSLP immunoglobulin, antigen binding protein, or fragment thereof as described herein) on their surface. Binding complexes with Kd<10⁻⁶M can be detected using SPR. In various embodiments, the SPR may be carried out at 20° C., 25° C., 30° C. or 370 C.

### Compositions

It will be appreciated that numbering of the residues in tezepelumab is based on the heavy chain and light chain variable sequences set out in SEQ ID NOs: 10 and 12, respectively, as well as the full length antibody heavy chain and light chain set out in SEQ ID NOs: 13 and 14, respectively.

In various embodiments, provided is a composition comprising tezepelumab and one or more tezepelumab derivatives, each comprising: a light chain CDR1 sequence comprising the amino acid sequence set forth in SEQ ID NO:3; a light chain CDR2 sequence comprising the amino acid sequence set forth in SEQ ID NO:4; a light chain CDR3 sequence comprising the amino acid sequence set forth in SEQ ID NO:5; a heavy chain CDR1 sequence comprising the amino acid sequence set forth in SEQ ID NO:6; a heavy chain CDR2 sequence comprising the amino acid sequence set forth in SEQ ID NO: 7; and a heavy chain CDR3 sequence comprising the amino acid sequence set forth in SEQ ID NO:8, wherein the derivatives comprise at least one of: isomerization derivatives, deamidation derivatives, oxidation derivatives, glycosylation derivatives, HMW species, fragments, disulfide isoform derivatives, or combinations thereof. In various embodiments, the composition comprises tezepelumab and one or more tezepelumab derivatives, each comprising a heavy chain amino acid sequence set out in SEQ ID NO: 10 and a light chain amino acid sequence set out in SEQ ID NO: 12.

Isomerization derivatives comprise alteration to aspartic acid residues. Exemplary isomerization at aspartic acid include isoaspartic acid (isoAsp), cyclic aspartate (cAsp), succinimide or other isomerization intermediates. The isomerization derivative in the composition may comprise a derivative in the heavy chain or light chain complementarity determining region (CDR) or within other parts of the variable region. In various embodiments, the isomerization is in the CDR. The isomerization derivative comprises a change at heavy chain CDR D54 of SEQ ID NO: 7, and/or light chain CDR D49, D50 or D52 of SEQ ID NO: 4, in either or both variable region chains. In various embodiments, the amount of the isomerization derivative in the composition is from about 0.5% to about 30%, or about 0.5% to 13%. In some embodiments, the composition comprises tezepelumab and derivatives thereof wherein isomerization at D54 is in an amount of less than about 5%, and/or wherein the isomerization at one or more of D49, D50 or D52 in an amount of less than about 13%. In various embodiments, the tezepelumab and tezepelumab derivatives have a greater potency and/or tolerability than a composition comprising greater than 30% of the isomerization derivative, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 30% of the isomerization derivative, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

Deamidation derivatives comprise alteration to asparagine residues. Exemplary deamidation derivatives include complete deamidation and deamidation intermediates. The deamidation derivative in the composition may comprise deamidated asparagine N25/N26 in LCDR1 set out in SEQ ID NO: 3, N316 in the heavy chain variable region set out in SEQ ID NO: 13, and/or N385/390 in the heavy chain variable region set out in SEQ ID NO: 13. In various embodiments, the composition comprises a deamidation derivative comprising deamidation at N25/N26 in an amount of less than about 3%, and/or deamidation at one or more of N316, and/or N385/390 in an amount of less than about 13%. In some embodiments, the amount of the deamidation derivative in the composition is between about 0.5%-10%. In various embodiments, the tezepelumab and tezepelumab derivatives have a greater potency and/or tolerability than a composition comprising greater than 15% of the deamidation derivative, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 15% of the deamidation derivative, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

Oxidation derivatives comprise alteration to one or more of methionine or tryptophan residues in the protein. Exemplary oxidation derivatives include complete oxidation or oxidation intermediates. The oxidation derivative in the composition may comprise oxidation at one or more of heavy chain methionine M34 of HCDR1 set out in SEQ ID NO: 6, or M253 or M359 in the heavy chain constant region set out in SEQ ID NO: 13, or heavy chain tryptophan W52 in HCDR2 set out in SEQ ID NO: 7, W90 of LCDR3 set out in SEQ ID NO: 5, or W102 in HCDR3 set out in SEQ ID NO: 8, in either or both heavy chains (or light chains, as applicable). For example, the oxidation derivative in the composition may comprise oxidation at one or more of heavy chain methionine M34 of HCDR1 set out in SEQ ID NO: 6, heavy chain tryptophan W52 in HCDR2 set out in SEQ ID NO: 7, light chain W90 of LCDR3 set out in SEQ ID NO: 5, or heavy chain W102 in HCDR3 set out in SEQ ID NO: 8 in either or both heavy chains (or light chains, as applicable). In various embodiments, the oxidation derivative comprises oxidation at one or more of heavy chain methionine M34, M253, M359, in either or both heavy chains, optionally wherein the oxidation is in an amount of less than about 7%. In various embodiments, the oxidation derivative comprises oxidation at one or more of tryptophan W52, W90, or W102 in either or both heavy chains, optionally wherein the oxidation is in an amount of less than about 7%, optionally less than about 5%, or less than about 3%. In some embodiments, the amount of the oxidation derivative in the composition is between about 0.4% to about 7%. In various embodiments, the tezepelumab and tezepelumab derivatives have a greater potency and/or tolerability than a composition comprising greater than 7% of the oxidation derivative, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 7% of the oxidation derivative, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

High Molecular Weight derivatives comprise aggregation of antibody, either into dimers or larger protein aggregates. HMW species contemplated herein include dimers and oligomers of tezepelumab. In various embodiments, the HMW species is a dimer. In various embodiments the dimers are covalently or non-covalently associated. In various embodiments, the amount of the HMW species in the composition is about 1.7% or less, about 1.6% or less, about 1.5% or less, about 1.4% or less, about 1.3% or less, about 1.2% or less, about 1.1% or less, about 1.0% or less, about 0.9% or less, about 0.8% or less, about 0.7% or less, about 0.6% or less, about 0.5% or less, or about 0.4% or less. In various embodiments, the tezepelumab and tezepelumab derivatives have a greater potency and/or tolerability than a composition comprising greater than 20% of the HWM species, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 20% of the HWM species, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

Tezepelumab fragment derivatives include protein products that may be cleaved by internal peptidases during production or produced by other steps in the production process. Tezepelumab fragments include low molecular weight (LMW) species, e.g., less than about 25kD, or middle molecular weight (MMW) species, e.g., between 25 to 50 kD, or combinations thereof. In various embodiments, the amount of the fragment in the composition is about 1.7% or less, about 1.6% or less, about 1.5% or less, about 1.4% or less, about 1.3% or less, about 1.2% or less, about 1.1% or less, about 1.0% or less, about 0.9% or less, about 0.8% or less, about 0.7% or less, about 0.6% or less, about 0.5% or less, or about 0.4% or less. In various embodiments, the tezepelumab and tezepelumab derivatives have a greater potency and/or tolerability than a composition comprising greater than 15% of the fragment species, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 15% of the fragment species, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

Glycosylation derivatives of tezepelumab comprise alteration of the profile of sugar residues that can be post-translationally applied to asparagine residues in the Fc region of an antibody. Exemplary glycosylation derivatives include afucosylation, application of galactosyl moieties (galactosylation) and application of high mannose moieties to asparagine. Glycosylation derivatives contemplated herein are changes to sugar residues at asparagine N298 in the Fc region set out in SEQ ID NO: 13, on one or both of the heavy chains. In various embodiments, the amount of glycosylation derivative in the composition is less than about 40%, about 35%, about 30%, about 25%, about 20%, about 15%, about 10% or about 5%. In some embodiments, the glycosylation derivative comprises afucosylated derivatives in an amount of less than about 5%, about 4%, about 3%, or about 2%. In various embodiments, the glycosylation derivative comprises galactosyl moieties in an amount of less than about 30%, about 25%, about 20%, about 15%, about 10% or about 5%. In various embodiments, the glycosylation derivative comprises high mannose moieties in an amount of about 25% or less, about 23% or less (e.g., about 23.1% or less), about 21% or less, about 19% or less, about 17% or less, about 15% or less, about 12% or less, about 10% or less, about 8% or less, about 5% or less, or about 4% or less. In various embodiments, the glycosylation derivatives comprise high mannose moieties in an amount of about 23.1% or less. In various embodiments, the glycosylation derivative comprises high mannose moieties in an amount of less than about 5%, about 4%, about 3%, about 2% or about 1%. In various embodiments, the tezepelumab and tezepelumab derivatives have a greater potency and/or tolerability than a composition comprising greater than 40% of the glycosylation derivatives, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 40% of the glycosylation derivatives, wherein said potency comprises a capability of inhibiting binding of TSLPR expressed on the surface of a Stat/BaF/HTR cell encoding a Stat luciferase reporter gene, the expression of which is indicative of binding of TSLP to TSLPR. In various embodiments, the tezepelumab and tezepelumab derivatives comprise no more than 15%, 13%, 11%, 8%, or 5% high mannose, and have less clearance (and/or a longer half-life) than a composition having greater than 15% high mannose. In various embodiments, the tezepelumab and tezepelumab derivatives comprise no more than about 25%, about 23%, about 21%, about 19%, about 17%, about 15%, about 13%, about 11%, about 8%, or about 5% high mannose, and have less clearance (and/or a longer half-life) than a composition having greater than about 25% high mannose. In various embodiments, the tezepelumab and tezepelumab derivatives comprise no more than about 23.1% high mannose, and have less clearance (and/or a longer half-life) than a composition having greater than about 23.1% high mannose. The percent high mannose may be determined by HILIC.

Tezepelumab or tezepelumab derivatives with “less clearance” refers to the amount of clearance from the body (blood or serum) being less when compared to the clearance of a reference antibody, e.g., tezepelumab or other IgG2 antibody. Clearance of tezepelumab or tezepelumab derivatives can be less than 1%, 2%, 3%, 4%, 5%, 6%, 7%, 8%, 9%, 10%, 11%, 12%, 13%, 14%, 15% or more of the clearance levels compared to the reference antibody. “Longer half-life” of tezepelumab or tezepelumab derivatives refers to the length of time the antibody is detectable in the body (blood or serum) being longer when compared to the half-life of a reference antibody, e.g., tezepelumab or other IgG2 antibody, in the body. The half-life of tezepelumab or tezepelumab derivatives can be 1%, 2%, 3%, 4%, 5%, 6%, 7%, 8%, 9%, 10%, 11%, 12%, 13%, 14%, 15% or longer than the half-life of the reference antibody.

In various embodiments, the tezepelumab and tezepelumab derivatives have a greater potency and/or tolerability than a composition comprising greater than about 40%, 35%, 30%, 25%, 23%, 21%, 20%, 19%, 184,17%, 16%, 15%, 14%, 13%, 12%, 11%, 10%, 9%, 8%, 7%, 6%, 5% or 4% of the glycosylation derivatives.

Potency and/or tolerability of glycosylation derivatives can also be associated with effector function and antibody clearance. In various embodiments, the tezepelumab and tezepelumab derivatives have less antibody clearance and/or greater tolerability than a composition comprising greater than about 15%, about 13%, about 11%, about 8% or about 6% high mannose glycosylation derivatives. In various embodiments, the tezepelumab and tezepelumab derivatives have less antibody clearance and/or greater tolerability than a composition comprising greater than about 25%, about 23%, about 19%, about 17%, about 15%, about 13%, about 11%, about 8% or about 6% high mannose glycosylation derivatives. In various embodiments, the tezepelumab and tezepelumab derivatives have less antibody clearance and/or greater tolerability than a composition comprising greater than about 23.1%, high mannose glycosylation derivatives.

Disulfide structural heterogeneity is inherent to recombinant and naturally occurring IgG2 molecules which contain 18 disulfide bonds—6 inter-chain and 12 intra-chain. The hinge:hinge peptides contain four disulfide linkages; in the classical IgG2-A structure. Unlike the classical IgG2-A structure, isomer IgG2-B contain symmetrical linkages connecting two copies of the Fab peptides (C_{H}1-C_{L}-hinge) with two copies of the hinge peptide. IgG2-A/B is an intermediate form, incorporating partial features of both IgG2-A and IgG2-B, defined by an asymmetrical arrangement involving one Fab arm covalently linked to two copies of the hinge peptide through disulfide bonds. Disulfide isoform derivatives comprise an IgG2-B isoform and/or an IgG2-A/B isoform. In various embodiments, the amount of the disulfide isoform derivative in the composition is less than about 75%. In some embodiments, when the derivative comprises comprise an IgG2-B isoform, the amount of the disulfide isoform derivative in the composition is less than about 20%, about 15%, about 10%, or about 5%. In some embodiments, when the derivative comprises an IgG2-A/B isoform, the amount of the IgG2-A/B isoform in the composition is less than about 75%, about 70%, about 65%, about 60%, about 55%, about 50%, about 45% or about 35%. In various embodiments, the amount of the IgG2-A/B isoform in the composition is from about 38% to about 43%. In various embodiments, the tezepelumab and tezepelumab derivatives have a greater potency and/or tolerability than a composition comprising greater than 75% of the disulfide isoform derivatives, wherein said potency comprises a capability of inhibiting binding of biotinylated TSLPR immobilized on a donor bead to TSLP-His immobilized on an acceptor bead. In various embodiments, the tezepelumab and tezepelumab derivatives have a greater potency and/or tolerability than a composition comprising greater than 75% of the disulfide isoform derivatives, wherein said potency comprises a capability of inhibiting binding of TSLPR expressed on the surface of a Stat/BaF/HTR cell encoding a Stat luciferase reporter gene, the expression of which is indicative of binding of TSLP to TSLPR.

In various embodiments, the composition has one or more of the following characteristics:
(a) the amount of isomerization derivatives in the composition is about 30% or less as measured by reduced peptide mapping;
(b) the amount of deamidation derivatives in the composition is about 15% or less as measured by peptide mapping;
(c) the amount of oxidation derivatives in the composition is about 7% or less as measured by reduced peptide mapping;
(d) the amount of glycosylation derivatives in the composition is about 40% or less as measured by glycan mapping;
(e) the amount of disulfide isoform derivatives in the composition is about 75% or less as measured bynon-reduced reversed phase high performance liquid chromatography (RP-HPLC);
(f) the amount of HMW species in the composition is about 20% or less as measured by SE-HPLC; and/or
(g) the amount of fragments in the composition is about 15% or less as measured by rCE-SDS.

In some embodiments, the composition is part of a formulation described herein. In some embodiments, the composition is a drug substance used to produce a formulation as described herein.

### Methods of Administration

In one aspect, methods of the present disclosure include a step of administering a therapeutic anti-TSLP antibody or antibody derivative described herein, optionally in a pharmaceutically acceptable carrier or excipient. In certain embodiments, the pharmaceutical composition is a sterile composition.

Contemplated herein are methods for treating an inflammatory disease, condition or disorder, such as asthma, chronic obstructive pulmonary disease (COPD), atopic dermatitis, eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF) with an anti-TSLP antibody or antigen binding protein or fragments thereof as described herein. In various embodiments, the disease, condition or disorder is asthma, including severe asthma, eosinophilic or non-eosinophilic asthma and low eosinophil asthma.

Asthma is a chronic inflammatory disorder of the airways. Each year, asthma accounts for an estimated 1.1 million outpatient visits, 1.6 million emergency room visits, 444,000 hospitalizations (Defrances et al, 2008) Available at: The Centers for Disease Control website, www.cdc.gov/nchs/data/nhsr/nhsr005.pdf, and 3,500 deaths in the U.S. In susceptible individuals, asthmatic inflammation causes recurrent episodes of wheezing, breathlessness, chest tightness, and cough. The etiology of asthma is thought to be multi-factorial, influenced by both genetic environmental mechanisms (To et al., BMC Public Health 2012; 12:204; Chung et al. Eur Respir J 2014; 43:343-73), with environmental allergens an important cause (Chung et al., supra; Pavord I D, et al., NPJ Prim Care Respir Med 2017; 27:17). The majority of cases arise when a person becomes hypersensitive to allergens (atopy). Atopy is characterized by an increase in Th2 cells and Th2 cytokine expression and IgE production. Approximately 10 million patients in the United States are thought to have allergy-induced asthma. Despite the available therapeutic options, asthma continues to be a major health problem. Worldwide, asthma currently affects approximately 300 million people; by 2020, asthma is expected to affect 400 million people (Partridge, Eur Resp Rev. 16:67-72, 2007).

Allergen inhalation by atopic asthmatics induces some of the manifestations of asthma, including reversible airflow obstruction, airway hyperresponsiveness, and eosinophilic and basophilic airway inflammation. Allergen inhalation challenge has become the predominant model of asthma in many species (Bates et al., Am J Physiol Lung Cell Mol Physiol. 297(3):L401-10, 2009; Diamant et al., J Allergy Clin Immunol. 132(5):1045-1055, 2013.)

Different asthma subtypes that are refractory to steroid treatment have been identified. Eosinophils are important inflammatory cells in allergic asthma that is characteristically mediated by Th2-type CD4+T cells. Neutrophilic airway inflammation is associated with corticosteroid treatment in severe asthma and can be mediated by Th1- or Th17-type T cells (Mishra et al., Dis. Model. Mech. 6:877-888, 2013).

Measures of diagnosis and assessment of asthma include the following: Airway inflammation evaluated using a standardized single-breath Fraction of Exhaled Nitric Oxide (FeNO)(American Thoracic Society; ATS, Am J Respir Crit Care Med. 171(8):912-30, 2005) test. Spirometry is performed according to ATS/European Respiratory Society (ERS) guidelines (Miller et al, Eur Respir J. 26(1):153-61, 2005). Post-bronchodilator (Post-BD) spirometry testing is assessed after the subject has performed pre-BD spirometry. Maximal bronchodilation is induced using a SABA such as albuterol (90 μg metered dose) or salbutamol (100 μg metered dose) or equivalent with a spacer device for a maximum of 8 total puffs (Sorkness et al, J Appl Physiol. 104(2):394-403, 2008). The highest pre- and post-BD FEV₁ obtained after 4, 6, or 8 puffs is used to determine reversibility and for analysis. Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV₁ (Juniper et al, Oct 1999). The ACQ-6 is a shortened version of the ACQ that omits the FEV₁ measurement from the original ACQ score. The mean ACQ score is the mean of the responses. Mean scores of ≤0.75 indicate well-controlled asthma, scores between 0.75 and ≤1.5 indicate partly-controlled asthma, and a score >1.5 indicates uncontrolled asthma (Juniper et al, Respir Med. 100(4):616-21, 2006). Individual changes of at least 0.5 are considered to be clinically meaningful (Juniper et al, Respir Med. 99(5):553-8, 2005). The Asthma Quality of Life Questionnaire, Standardized (AQLQ[S])+12 (AQLQ(S)+12) is a 32-item questionnaire that measures the HRQoL experienced by asthma patients (Juniper et al, Chest. 115(5):1265-70, May 1999). The Asthma Daily Diary is also used for assessment.

Related US Patent Publication US-2018-0296669 (incorporated herein by reference) discloses that treatment with an anti-TSLP antibody is effective at reducing asthma symptoms in a no eosinophil/low eosinophil population as it is in a high eosinophil population. Also contemplated is a method of reducing the frequency of asthma exacerbation in a subject.

Also contemplated herein are methods of treating asthma in a subject having a Th2 high asthma profile or a Th2 low asthma profile. It is contemplated that a TSLP antagonist that inhibits binding of the TSLP protein to its receptor complex will effectively treat a low eosinophil asthma population as the antibody described herein. Similarly, it is contemplated that a TSLP antagonist that inhibits binding of TSLP to its receptor complex will be effective in treating Th2 low asthma populations. Also contemplated are methods for treating chronic obstructive pulmonary disease (COPD) in a subject comprising administering an anti-TSLP antibody or antibody derivative or antigen binding protein described herein. It is contemplated that the subject to be treated is human. The subject may be an adult, an adolescent or a child.

Therapeutic antibody (or antibody derivative) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the anti-TSLP antibody or antibody derivative is administered in a dose range of about 70 mg to about 280 mg per daily dose. For example, the dose may be given in about 70 mg, 210 mg or 280 mg. In various embodiments, the anti-TSLP antibody or antibody derivative may be administered at a dose of 70, 80, 90, 100, 110, 120, 130, 140, 150, 160, 10, 180, 190, 200, 210, 220, 230, 240, 250, 260, 270 or 280 mg per dose. These concentrations may be administered as a single dosage form or as multiple doses. The above doses are given every two weeks or every four weeks. In various embodiments, the anti-TSLP antibody or antibody derivative is administered at a single dose of 70 mg every two weeks or every four weeks. In various embodiments, the anti-TSLP antibody or antibody derivative is administered at a single dose of 210 mg every two weeks or every four weeks. In various embodiments, the anti-TSLP antibody or antibody derivative is administered at a single dose of 280 mg every two weeks or every four weeks.

For antibody derivatives, the amount of antibody derivative should be such that the number of TSLP binding sites that are in the dose have an equimolar number of TSLP binding sites to canonical bivalent antibody described above.

It is contemplated that the anti-TSLP antibody or antibody derivative is administered every 2 weeks or every 4 weeks for a period of at least 4 months, 6 months, 9 months, 1 year or more. In various embodiments, the administration is subcutaneous or intravenous.

Treatment with the anti-TSLP antibody or antibody derivative is contemplated to decrease eosinophils in blood, sputum, broncheoalveolar fluid, or lungs of the subject. It is also contemplated that the administration shifts cell counts in the subject from a Th2 high population to a Th2 low population. It is further contemplated that administration of the anti-TSLP antibody improves one or more measures of asthma in a subject selected from the group consisting of forced expiratory volume (FEV), FEV1 reversibility, forced vital capacity (FVC), FeNO, Asthma Control Questionnaire-6 score and AQLQ(S)+12 score.

Improvement in asthma may be measured as one or more of the following: reduction in AER (annualized exacerbation rate), reduction in hospitalizations/severe exacerbations for asthma, change from baseline (increase) in time to first asthma exacerbation (following onset of treatment with anti-TSLP antibody), decrease relative to placebo in proportion of subjects with one or more asthma exacerbations or severe exacerbations over the course of treatment, e.g., 52 weeks, change from baseline (increase) in FEV1 and FVC (pre-broncholdilator and post-bronchodilator), change from baseline (decrease) in blood or sputum eosinophils (or lung eosinophils if biopsy or BAL fluid obtained), change from baseline (decrease) in FeNO, change from baseline (decrease) in IgE, improvement in asthma symptoms and control as measured by PROs including ACQ and variants, AQLQ and variants, SGRQ, and asthma symptom diaries, change (decrease) in use of rescue medications, decrease in use of systemic corticosteroids, decrease in Th2/Th1 cell ratio in blood. Most/all these measures should be in total population and subpopulations including hi and low eosinophils (Greater than or equal to 250 is high; less than 250 is low), allergic and non-allergic, Th2 hi and low, Periostin hi and low (compared to median value), and FeNO hi and low (greater than or equal to 24 or less than 24).

Also contemplated in the present disclosure is the administration of multiple agents, such as an antibody composition in conjunction with a second agent as described herein, including but not limited to an anti-inflammatory agent or asthma therapy.

However, it is contemplated that, in various embodiments, the administration reduces frequency of or levels of co-administered therapy in the subject. Exemplary co-administered therapies include, but are not limited to, inhaled corticosteroids (ICS), long-acting β2 agonist (LABA), leukotriene receptor antagonists [LTRA], long-acting anti-muscarinics [LAMA], cromones, short-acting β2 agonist (SABA), and theophylline or oral corticosteroids. In various embodiments, the administration eliminates the need for corticosteroid therapy.

### Formulations

In some embodiments, the disclosure contemplates use of pharmaceutical compositions comprising a therapeutically effective amount of an anti-TSLP antibody or antibody derivative together with a pharmaceutically acceptable diluent, carrier, solubilizer, emulsifier, preservative, and/or adjuvant. In addition, the disclosure provides methods of treating a subject by administering such pharmaceutical composition.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor.

The formulation components are present preferably in concentrations that are acceptable to the site of administration. In certain embodiments, buffers are used to maintain the composition at physiological pH or at a slightly lower pH, typically within a pH range of from about 4.5 to about 8. Including about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

In various embodiments, the anti-TSLP antibody or antibody derivative is in a formulation containing acetate, and one or more of proline, sucrose, polysorbate 20 or polysorbate 80. In various embodiments, the formulation comprises 5-50 mM acetate, less than or equal to 3% (w/v) proline, 0.015% (w/v)±0.005% (w/v) polysorbate 20 or polysorbate 80, at pH between 4.9 and 6.0. Optionally, the antibody or antibody derivative is at a concentration of between about 100 and about 150 mg/ml. The formulation may be stored at −20° to −70° C. Exemplary anti-TSLP formulations comprising these excipients are described in International Application No. PCT/US2021/018561, herein incorporated by reference.

In alternative embodiments, the anti-TSLP antibody or antibody derivative is in a formulation containing a surfactant, and at least one basic amino acid or a salt thereof. In exemplary instances, the basic amino acid is arginine or histidine. In various embodiments, the salt is arginine glutamate or histidine glutamate, optionally in a concentration of from 10 to 200 mM. Optionally, the formulation further comprises proline. In alternative embodiments, the anti-TSLP antibody or antibody derivative is in a formulation containing a surfactant, and calcium or a salt thereof. In various embodiments, the salt is calcium glutamate, optionally in a concentration from 15 mM to about 150 mM. Optionally, the formulation further comprises proline. In various embodiments, the surfactant is polysorbate 20 or polysorbate 80 or a mixture thereof. Optionally, the antibody or antibody derivative is at a concentration of greater than about 110 mg/ml, or greater than about 140 mg/ml. Exemplary anti-TSLP formulations comprising these excipients are described in International Patent Application No. PCT/US2021/017880, herein incorporated by reference.

When parenteral administration is contemplated, the therapeutic compositions for use may be provided in the form of a pyrogen-free, parenterally acceptable aqueous solution comprising the desired anti-TSLP antibody or derivative thereof in a pharmaceutically acceptable vehicle. A particularly suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody. In various embodiments, the administration may be via pre-filled syringe or autoinjector. In various embodiments, the auto-injector is an Ypsomed YpsoMate®. In various embodiments, the auto-injector is disclosed in WO 2018/226565, WO 2019/094138, WO 2019/178151, WO 20120/072577, WO2020/081479, WO 2020/081480, PCT/US20/70590, PCT/US20/70591, PCT/US20/53180, PCT/US20/53179, PCT/US20/53178, or PCT/US20/53176.

### Kits

As an additional aspect, the disclosure includes kits which comprise one or more compounds or compositions packaged in a manner which facilitates their use to practice methods of the disclosure. In one embodiment, such a kit includes a compound or composition described herein, packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the antibody composition.

Additional aspects and details of the disclosure will be apparent from the following examples, which are intended to be illustrative rather than limiting.

### EXAMPLES

### Example 1—Identification of Tezepelumab Attributes

Tezepelumab is a full-length, human monoclonal antibody of the IgG2 subclass produced in Chinese Hamster Ovary (CHO) cells. It consists of 2 heavy chains (HC) and 2 light chains (LC) of the lambda subclass. The heavy and light chains are covalently linked through disulfide bonds. Biochemical, biophysical, and biological characterization of tezepelumab was conducted to provide a comprehensive understanding of its structural and functional properties and to enable an assessment of antibody attributes that may affect binding and potency.

### Materials and Methods

AMG 157 and labile residues potentially impacting binding: Amino acid sequence of AMG157 as sequence A5 (and as chains H5, L5) and also several other TSLP-binding antibodies were previously described in U.S. Pat. No. 7,982,016 B2.

Molecular mass of the antibody with A2GOF/A2GOF glycosylation (C6500 H9998 02068 N1734 S52) is 147189.4 Da, including heavy chain N-terminal pyroglutamate and C-terminal K removed. TSLP contained 74% monomeric, 23% dimeric and 3% tetrameric species.

Peptide Mapping: Peptide mapping of tezepelumab samples was performed using the sample preparation procedure including refolding with guanidine, reduction and alkylation of disulfide bonds, buffer exchange and digestion with trypsin on peptides suitable for LC-MS analysis as described in (Ren et al., Anal. Biochem. 392: 12-21 (2009)). Briefly, a sample comprising tezepelumab was diluted to about 1 mg/ml in 0.5 ml of pH 7.5 denaturation buffer (7.5 M guanidine hydrochloride (GdnHCI) and 0.25 M Tris). Reduction was accomplished with the addition of 3 μl of 0.5 M dithiothreitol (DTT) followed by 30 min of incubation at room temperature. Carboxy-methylation was achieved with the addition of 7 μl of 0.5 M iodoacetic acid (IAA). The reaction was carried out in the dark for 15 min at room temperature. Excess IAA was quenched with the addition of 4 μl of 0.5 M DTT. Reduced and alkylated tezepelumab samples were buffer-exchanged into a pH 7.5 digestion buffer (0.1 M Tris or 0.1 M ammonium bicarbonate) using a NAP-5 column (GE Healthcare, Piscataway, NJ, USA). Lyophilized trypsin was dissolved in water to a final concentration of 1 mg/ml. Digestion was started with the addition of the 1-mg/ml trypsin solution to the reduced, alkylated, and buffer-exchanged tezepelumab samples to achieve a 1:25 enzyme/substrate ratio. Digestion was carried out at 37° C. for 30 min. The final digest was quenched with the addition of 5 tl of 20% FA. LC-MS/MS peptide mapping analysis of the digested tezepelumab samples was performed on an Agilent 1290 UHPLC system connected to a Thermo Scientific Q-Exactive Biopharma mass spectrometer as described in (Ren et al., 2009, supra). Acquired LC-MS/MS raw data and sequences of tezepelumab and target were used to identify and quantify modifications by MassAnalyzer software (Zhang, Anal. Chem. 81: 8354-8364 (2009)).

SE-UHPLC: Tezepelumab sample were loaded onto an analytical SE-UHPLC column (BEH200 column, 1.7 μm particle size, 4.6 mm×150 mm, Waters Corporation) and proteins were separated isocratically using a mobile phase comprising 100 mM sodium phosphate, 250 mM sodium chloride at pH 6.8. The eluent was monitored by UV absorbance at 280 nm. The column was operated at ambient temperature and the mobile phase was applied to the column at a flow rate of 0.4 mL/min.

Non-Reduced RP-HPLC: Tezepelumab samples were analyzed by RP-HPLC using a Waters BEH300 C4 column (1.7 μm particle size, 2.1 mm×50 mm) and eluted using a 0.1% TFA-containing mobile phase and a gradient of 1-propanol at 75° C. Absorbance at 215 nm was monitored.

Reduced CE-SDS: Tezepelumab samples are analyzed by rCE-SDS. Samples were reduced and denatured by heating in the presence of sodium dodecyl sulfate (SDS) and p-mercaptoethanol at pH 6.5 prior to electrokinetic injection into a bare-fused silica capillary filled with SDS gel buffer at 25° C. Absorbance was monitored at 220 nm.

CEX-UHPLC: Samples of tezepelumab drug substance were loaded onto an analytical CEX-HPLC column (BioPro SP-F, 5 μm particle size, 4.6 mm×100 mm, YMC America, Inc.). Mobile phase A contained 20 mM sodium phosphate at pH 6.6 and mobile phase B consisted of 20 mM sodium phosphate, 500 mM sodium chloride, at pH 6.6. Proteins were separated using a linear salt gradient generated with 5% to 12% mobile phase B from 0 min to 4 min, to 23% mobile phase B at 18 min, to 100% mobile phase B at 18.5 min to 20.5 min, and back to 5% mobile phase B at 21 min to 25 min. The eluent was monitored by UV absorbance at 280 nm. The column was operated at 28° C. and the mobile phase was applied to the column at a flow rate of 0.6 mL/min.

Glycan Mapping: N-glycan mapping is an analytical technique in which oligosaccharides attached to asparagine residues are released through enzymatic cleavage. Free oligosaccharides are subsequently derivatized with a fluorescent tag for detection and quantitation. The labeled oligosaccharides are resolved by hydrophilic interaction liquid chromatography (HILIC) with fluorescence detection to generate a glycan profile. In this method, Tezepelumab is subjected to enzymatic digestion with N-glycosidase F (PNGase F), which specifically cleaves the bond between the N-acetylglucosamine (GIcNAc) of the oligosaccharide and the asparagine residue. The released oligosaccharides are labeled with 2-Aminobenzoic Acid (2-AA) via reductive amination. Following a centrifugation cleanup step, the oligosaccharides are separated by HILIC on an ultra-performance liquid chromatography (UPLC) system. The relative % peak areas of the major oligosaccharide species are calculated.

Potency: Potency of the tezepelumab compositions comprising attributes described herein was observed by a receptor-ligand binding bioassay and/or a reporter gene cell-based bioassay.

Receptor-Liqand Binding Assay: This assay provides a proximal measure of tezepelumab activity and directly reflects the molecular mechanism of action of tezepelumab, which is to bind TSLP and prevent it from binding to the TSLP receptor (TSLPR). This method provides a quantitative measure of the ability of tezepelumab to inhibit the binding of TSLP to TSLPR. Tezepelumab binds to the recombinant TSLP-His ligand (TSLP-His) and inhibits it from binding to biotinylated TSLP Receptor (TSLPR). The potency assay is a bead-based Amplified Luminescent Proximity Homogeneous Assay (Alpha) that detects biomolecular interactions. The assay contains two bead types: acceptor beads and donor beads. The donor beads are coated with a hydrogel that contains phthalocyanine, a photosensitizer and streptavidin. The acceptor beads are coated with a hydrogel that contains thioxene derivatives as well as nickel chelate. The donor beads bind to biotinylated TSLPR through interaction between streptavidin and biotin, and the acceptor beads bind to histidine tagged TSLP due to the interaction between nickel chelate and histidine. When TSLP-His and biotinylated TSLPR bind to each other, the acceptor beads and the donor beads are brought into close proximity. When a laser is applied to this complex, ambient oxygen is converted to singlet oxygen by the donor beads. If the beads are in close proximity, an energy transfer to the acceptor beads occurs, resulting in the production of luminescence, which is measured in a plate reader equipped with AlphaScreen® signal detection capabilities. Tezepelumab binds to TSLP-His and prevents it from binding to biotinylated TSLPR, thereby decreasing the luminescence output in a dose dependent manner. The test sample activity is determined by comparing the test sample response to the response obtained for the Reference Standard. It will be appreciated that the Receptor-Ligand Binding Assay described in this paragraph is a suitable assay for determining the capability of a composition to inhibit binding of biotinylated TSLPR immobilized on a donor bead to TSLP-His immobilized on an acceptor bead.

Cell-Based Reporter Gene Bioassay: Human Thymic Stromal Lymphopoietin (TSLP) protein binding to human TSLP receptor (TSLPR) complex expressed on the surface of stable murine BaF cells induces Stat 5 activation and cell proliferation. This method utilizes the murine BaF/hu HTR cell line that were co-transfected with plasmids encoding the Stat luciferase reporter gene and blasticidin-resistant gene. When Stat/BaF/HTR cells are incubated with recombinant human TSLP, signal transduction occurs following binding to the TSLPR, resulting in the increase of luciferase activity. AMG 157 antagonizes TSLP induced activity of the TSLPR, thus inhibiting TSLP mediated luciferase response. This method measures the dose dependent inhibitory effect of AMG 157 Reference Standard and test samples on Stat/BaF/HTR cells stimulated with TSLP. Following incubation with TSLP and tezepelumab, the cells are treated with a reagent containing a detergent (for cell lysis) and luciferin, a substrate for luciferase. The reaction of luciferase with luciferin results in luminescence that is measured in a luminometer. Production of luciferase in reporter cells in response to TSLP stimulation is quantified by luminescence reading after addition of luciferase substrate. The degree of inhibition of TSLP induced activation of luciferase reporter activity is proportional to the amount of tezepelumab. Test sample biological activity is determined by comparing the test sample response to the Reference Standard. It will be appreciated that the Cell-Based Reported Gene Assay described in this paragraph is a suitable assay for determining the capability of a composition to inhibit binding of TSLPR expressed on the surface of a Stat/BaF/HTR cell encoding a Stat luciferase reporter gene, the expression of which is indicative of binding of TSLP to TSLPR.

### Results

Biochemical characterization of tezepelumab identified modified tezepelumab antibodies that could be isolated from tezepelumab preparations and after storage of drug substance including isomerization derivatives, deamidation derivatives, oxidation derivatives, high molecular weight species, fragmented species, partially reduced species, high mannose glycan derivatives, or disulfide isoform derivative. These attributes were assessed for their potential impact on potency and tolerability of tezepelumab.

Isomerization: Aspartic acid isomerization was assessed by reduced peptide mapping with LC-MS/MS. Aspartic acid residues, whether native or formed by deamidation of Asn, may undergo isomerization via a cyclic imide intermediate. The isomerization may impact target binding and efficacy based on a modified residue's proximity to the CDR. Native levels of isomerization in tezepelumab were assessed by mass spectrometric analysis of peptide mapping studies. Isomerization at HC CDR2 Asp⁵⁴ and LC CDR3 Asp^{91/95} was not observed at significant level in drug substance. Thermal exposure forced degradation studies showed LC CDR2 Asp^{49/50}to be sensitive to isomerization at elevated temperatures. In addition, isomerization levels of HC CDR2 Asp⁵⁴ also showed minor increase (<2%) at elevated temperature. Therefore, the predominant isomerization sites were identified as LC CDR Asp^{49/50} and HC CDR Asp⁵⁴ at 10% and 2% after 5 weeks of thermal forced degradation (40° C.).

Isomerization of Asp^{49/50} in the light chain CDR2 was observed at approximately 0.2% in drug substance. Isomerization at HC CDR2 Asp⁵⁴ and LC CDR3 Asp^{91/95} was not observed at significant level in drug substance.

Product lots used in clinical trials at end of shelf life were monitored for levels of impurities such as HMW species, fragments, isomerization, etc., and compared with the impurities at the initial release of the same lot. An increase in impurities over time allows for calculation of clinical exposure of subject to levels of impurities and determination of the effects of these attributes on product safety, and provides a measure of tolerability of impurities in the drug substance. For example, a clinical study of tezepelumab utilized drug substance which was dosed up to the last month of its 36 month clinical shelf life. The use of aged drug product combined with the higher and more frequent dosing exposed patients to higher levels of product related substances and product related impurities than with new drug product lots. The elevated exposure was primarily by virtue of the increased cumulative dosing per month of treatment (e.g., via 420 mg Q14D subcutaneous injection dosing), compared to a dose of 210 mg monthly subcutaneous injection dosing.

The clinical dosing of 420 mg every two weeks by subcutaneous injection is approximately 4× greater than the 210 mg monthly dose. From this dosing scheduled, it was calculated that systemic exposure at the higher dose regimen as expressed by either the “area under the curve” (AUC) or maximum serum concentration (Cₘₐₓ) is 3.2× to 3.7× greater, respectively, than the lower clinical dose. Per the clinical study protocol, antibody testing would be performed only if there was an unexpected change in exposure or potentially anti-drug antibody-related safety events. These outcomes were not observed and the drug was well tolerated.

Based on dosage and estimated attribute levels at time of administration in this clinical trial, levels of attribute exposure to patients of the clinical trial were estimated and tolerability assessed. For example, the % attribute in the drug product (e.g., HWM) can be multiplied by the clinical exposure multipliers to determine the equivalent % attribute levels in a product lot administered at the proposed dose of 210 mg Q28D.

For isomerization, calculated total isomerization of up to 30%, based on a dose 210 mg Q28D, was not associated with any safety-related issues in vivo. Calculated isomerization D49/D50 or D52 of 26% based on a dose of 210 mg Q28D was not associated with any safety issues in vivo, whereas calculated isomerization of D54 of 4% based on a dose of 210 mg Q28D was not associated with any safety issues in vivo.

Oxidation: Oxidation was assessed using reduced tryptic peptide map LC-MS. Oxidation at a methionine (Met) residue is a post-translational modification that can potentially arise as a result of exposure to oxygen and/or chemical oxidizing agents, as well as photo exposure. Tezepelumab contains 8 Met residues in each heavy chain (Met², Met³⁴, Met⁸³, Met¹¹⁷, Met²⁵³, Met³⁵⁹, Met³⁹⁸, Met⁴²⁹). There are no Met residues in the light chain. Only one Met residue, Met³⁴, is located in the complementarity determining region (CDR). Low but detectable levels of oxidation were observed at residues Met², Met¹¹⁷, Met²⁵³, Met³⁵⁹, Met³⁹⁸ of heavy chain (Table 1). Methionine oxidation in CDR can potentially impact potency, however, oxidation at Met³⁴ from CDR region was not observed. The degree of oxidation was estimated by reduced peptide map with mass spectrometric detection (ESI-MS). Inference from the relative intensity of the oxidized and unoxidized species was used to calculate the relative percentage; however, this approach is considered semi-quantitative due to potential differences in ionization efficiencies and co-elution of interfering species. Analysis of tezepelumab under forced oxidation was used to elucidate the susceptibility of specific sites on the molecule to oxidation.

TABLE 1
Methionine Oxidation Levels in Tezepelumab Drug Substance

| Residue | Oxidation | Loop (Element) |
|---|---|---|
| Met² | ~1% | V_{H} (Fab) |
| Met¹¹⁷ | ~1% | CH₁ (Fab) |
| Met²⁵³ | ~2% | CH₂ (Fc) |
| Met³⁵⁹ | ~3% | CH₂ (Fc) |
| Met³⁹⁸ | ~1% | CH₃ (Fc) |
| Met⁴²⁹ | <1% | CH₃ (Fc) |

Forced chemical oxidation showed that the order of sensitivity for heavy chain methionine is Met¹¹⁷>Met²⁵³>Met²>Met⁴²⁹ indicating that Met¹¹⁷ and Met²⁵³ are the sites with the greatest solvent exposure. Similar to the chemical oxidation studies above, photo degradation studies established the relative sensitivities of heavy chain Met residues to light induced oxidation as follows: Met²⁵³>Met¹¹⁷>Met³⁹⁸>Met³⁵⁹. The level of Met34 oxidation is below the level of quantitation, and given the sequence and molecule folding suggests this residue is not available for oxidation. In addition, the photo degradation studies showed increase in oxidation levels of tryptophan residues in the order of Trp¹⁰²>Trp⁵⁶>Trp⁵²>Trp⁹⁰, indicating heavy chain variable region Trp¹⁰² and light chain Trp⁵⁶ are the sites with the greatest light exposure (Table 2).

TABLE 2
Tryptophan Oxidation Levels in Tezepelumab Drug Substance

| Residue | Oxidation | Loop (Element) |
|---|---|---|
| Trp⁵⁶ | <1% | V_{L} (Fab) |
| Trp¹⁰² | <1% | V_{H} (Fab, CDR3) |
| Trp¹⁵⁰ | <1% | CH₁ (Fab) |
| Trp²⁷⁸ | ~2% | CH₂ (Fc) |

Observed oxidation at end of shelf life EOS (maximum value 36 months 2-8C plus 2 months 30 C) showed oxidation in HCDR at W52 of approximately 0.2%, and oxidation at W102 of 1.1%. Drug substance oxidation detected 0.3 to 0.5% oxidized W102. Based on dosage and estimated attribute levels at time of administration in a human clinical trial, levels of attribute exposure to patients of the clinical trial were estimated. Calculated oxidized W102 of up to 6-7% based on a dose of 210 mg Q28D was not associated with any safety issues in vivo. Tryptophan oxidation can occur at elevated temperature and under extreme visual and UV light exposure. CDR tryptophan oxidation caused by extreme conditions is associated with a moderate reduction in potency. There is a strong correlation between tryptophan oxidation and yellow color index.

Deamidation: Asparagine deamidation was assessed using tryptic peptide mapping with LC-MS. Native levels of deamidation in tezepelumab were assessed by ESI-MS/MS analysis of peptide mapping studies. Only low levels of deamidation were observed at residues Asn³¹⁶ and Asn³⁸⁵ of the heavy chain (Table 3). No deamidation at other sites, including Asn⁵⁷ and Asn^{25/26} in the heavy chain CDR2 and light chain CDR1, respectively, were observed in the drug substance.

TABLE 3
Asparagine Deamidation Levels in Tezepelumab Drug Substance

| Residue | Deamidation | Loop (Element) |
|---|---|---|
| Asn³¹⁶ | <1% | C_{H}2 (Fc) |
| Asn³⁸⁵ | 2% | C_{H}3 (Fc) |

Analysis of tezepelumab under forced deamidation conditions was used to evaluate the susceptibility of specific molecule sites to deamidation. At physiological pH 7.4, the most susceptible sites were determined to be Asn³⁹⁰ and Asn³⁸⁵ (Drug substance 3%; EOS, 5%), while a minor site was identified at Asn316 (Drug substance 0.09-0.1%; EOS, 0.4%), all of which are located in the Fc region. Deamidation in the LC variable CDR region at Asn²⁵ was only observed at a low level (Drug Substance, 0.1 to 0.2%; EOS, 0.4%). Based on dosage and estimated attribute levels at time of administration in a human clinical trial, levels of attribute exposure to patients of the clinical trial were estimated. Calculated deamidation at Asn25 up to 2% (based on a dose of 210 mg Q28D) was not associated with any safety issues in vivo, and calculated deamidation at Asn385/390 of up to 13% (based on a dose of 210 mg Q28D) was not associated with any safety issues in vivo.

Glycosylation: Glycosylation is relevant for antibody effector function and binding of antibody to Fc receptors on the surface of cells and altered glycosylation can interfere with one or more of these function. Effector function includes Antibody-Dependent Cell-mediated Cytotoxicity (ADCC) and Antibody-Dependent Cellular Phagocytosis (ADCP).

Tezepelumab is expected to contain a single N-glycosylation site at Asn²⁹⁸ on each heavy chain based on the presence of a consensus sequence, as well as historical characterization of IgG2 monoclonal antibodies produced from mammalian cell culture. The glycosylation sites were assessed by comparison of trypsin peptide maps with and without PNGaseF treatment. PNGaseF cleaves high mannose, hybrid, and complex glycan moieties between the reducing end N-acetylglucosamine residue of the glycan and the Asn residue of the peptide backbone. The compositions of species in the N-linked glycan map were determined by coupling the outlet from the chromatographic separation to an Orbitrap mass spectrometer.

Comprehensive characterization of the glycan complement of tezepelumab demonstrates the presence of biantennary N-linked structures with varying degrees of terminal galactosylation as predominant species and high levels (approximately 95%) of fucosylation. The glycan distribution of each of CEX drug substance, as determined by HILIC, is shown in Table 4.

TABLE 4
Glycan Peak Area % of Tezepelumab Drug Substance (DS)

| | Fucosylated | High Mannose | β-Galactosylated | Sialylated |
|---|---|---|---|---|
| Sample | (%) | (%) | (%) | (%) |
| DS | 93.8 | 3.9 | 22.8 | 0.1 |

The tezepelumab glycan derivative population contains galactosylated species (DS 19.9-28.6%), afucosylated species (DS 1.1-1.2%) and 3.9-4.8% high mannose species in the DS (predominantly as oligomannose 5). Based on dosage and estimated attribute levels at time of administration in a human clinical trial, levels of attribute exposure to patients of the clinical trial were estimated. Calculated afucosylated species of up to approximately 5%, calculated galactosylated species of up to 75-90%, and calculated high mannose derivatives of up to 14 to 18% were not associated with any safety issues in vivo, all estimates based on a dose of 210 mg Q28D.

To investigate the biological effect of N-linked glycan removal, tezepelumab was treated with PNGaseF, purified, and tested by the receptor-ligand binding assay and the cell-based reporter gene bioassay (Table 5). These results demonstrate that the removal of the N-glycans has no effect on tezepelumab in either potency assay.

TABLE 5
Biological Activity of Deglycosylated Tezepelumab

| | Receptor-Ligand | Cell-based Reporter |
|---|---|---|
| | Binding Assay | Gene Bioassay |
| Assessment Condition | % Relative Potency* | % Relative Potency* |
| Deglycosylated | 112 | 98 |

*Mean value of 3 replicates

Levels of high mannose glycans may potentially impact product half-life and process conditions for the production bioreactor can influence high mannose levels. The impact of production bioreactor process parameters on high mannose were evaluated in process characterization studies with pH identified as the main process parameter impacting high mannose. The acceptable range for pH was established to support consistent high mannose levels. High mannose levels in the process characterization studies were s 7.5%.

### Example 2—Size Derivatives

In addition to chemical changes at the amino acid level of tezepelumab, derivatives having aggregates or fragments are also possible.

The total expected mass of the peptide backbone of intact tezepelumab, assuming the presence of 2 unmodified light chains, 2 N-terminal pyroglutamidated heavy chains and 18 disulfides is 144,298 Da. In addition, native tezepelumab contains a single N-linked glycosylation site at Asn²⁹⁸ on each of the two heavy chains. The theoretical mass of intact, glycosylated tezepelumab with 2 copies of the glycan on Asn298, 2 copies of the predominant heavy chain C-terminal Gly derivative, and 2 copies of the predominant heavy chain N-terminal pyroglutamine is 147,189 Da. Treatment of intact tezepelumab with PNGaseF removes the N-glycosylation with a concomitant increase of 1 Da per chain from the polypeptide mass due to the conversion of the glycan-bearing Asn to an Asp residue and reduces the heterogeneity of the deconvoluted mass spectrum. The theoretical mass for deglycosylated material is 144,300 Da.

Size heterogeneity is an intrinsic property of proteins through the action of chemical or enzymatic cleavage, as well as self-association through various mechanisms. Potential size derivatives may include: High molecular weight (HMW) species through self-association to form species larger than monomer (dimer, higher order oligomeric species). HMW may be formed through non-covalent association, reducible covalent association, and/or non-reducible covalent association; Low molecular weight (LMW) species through truncation of the polypeptide backbone and/or incomplete assembly of subunit constituents (i.e., light chain and heavy chain.

Size heterogeneity of tezepelumab was evaluated using the following analytical methods: Size exclusion ultra-high performance liquid chromatography (SE-UHPLC) to assess size and purity under native conditions; Sedimentation velocity ultracentrifugation (SV-AUC) and SE-HPLC with static light scattering (SLS) detection to provide an additional assessment of molar mass; Reduced sodium dodecyl sulfate capillary electrophoresis (rCE-SDS) to determine size and purity under reducing and denaturing conditions. Derivatives separated include fragments, non-reducible covalent linkages, polypeptides lacking normal glycosylation or containing additional glycosylation sites; Non-reduced sodium dodecyl sulfate capillary electrophoresis (nrCE-SDS) to determine size and purity under denaturing conditions. Derivatives separated include partially assembled molecules, fragments, covalent linkages.

The results of these analytical techniques indicated that: Tezepelumab drug substance is predominantly composed of monomer, with low levels of dimer and LMW species, based on SE-UHPLC, SE-HPLC-SLS, and sedimentation velocity analytical ultracentrifugation (SV-AUC) results. Low levels of LMW species are observed under denaturing (nrCE-SDS) conditions and reduced and denaturing conditions (rCE-SDS). Based on rCE-SDS results, tezepelumab reduces to predominantly HC and LC components with minor levels of fragmented and HMW species. These include LMW (smaller than LC), middle molecular weight (MMW, smaller than HC, but larger than LC), and HMW (larger than HC) species. LMW species (e.g., less than 25 kD) and MMW species (between about 25 to 50 kD) were detected collectively as fragments and observed in less than 2% of in the tezepelumab preparation [DS: <0.4% (98.7-99% for HC+LC); EOS: 1.5% (97.3-97.5%% for HC+LC)].

Size heterogeneity of tezepelumab is monitored by non-denaturing SE-UHPLC, which is an in-process control method and a part of the drug substance and drug product release and stability testing program. The method is performed under non-denaturing conditions to resolve HMW species from monomer main peak. Tezepelumab drug substance was analyzed by SE-UHPLC on a Waters BEH200 4.6×150 mm 1.7 mm particle size column in mobile phase 100 mM sodium phosphate, 250 mM sodium chloride, pH 6.8 at a flow rate of 0.4 mL/min and detection at 280 nm absorbance. profile is dominated by the presence of a main peak (relative percent area 99.6%), eluting at approximately 2.8 minutes. A minor peak, best observed in the 20× enhanced chromatogram, elutes before the main peak at approximately 2.2 minutes retention time. This peak contains tezepelumab HMW and has a relative area percent of 0.4%, as shown in Table 6.

TABLE 6
Peak Area Percentage of Tezepelumab
Drug Substance by SE-UHPLC

| Peak Identification | Relative Area % |
|---|---|
| Main Peak | 99.6 |
| HMW | 0.4 |

Overall HMW species were detected in drug substance (DS) at approximately 0.3-0.6% but at EOS at 1.7%, e.g., ≤1.4% HMW (release) and ≤1.7 HMW (Stability). Based on dosage and estimated attribute levels at time of administration in a human clinical trial, levels of attribute exposure to patients of the clinical trial were estimated. Calculated HMW species of up to 20% HMW (based on a dose of 210 mg Q28D) were not associated with any safety related issues in vivo.

rCE-SDS was used to evaluate the heavy chain and light chain as well as LMW and MMW species. The rCE-SDS electropherogram for tezepelumab is presented in peak area % values shown in Table 7. These data demonstrate that tezepelumab is composed of disulfide-linked heavy chain and light chain. In tezepelumab drug substance, the minor peaks observed in the LMW and MMW regions are within the baseline noise and variability of the method. Consistent with SE-UHPLC results, almost no LMW or MMW species are observed. Based on dosage and estimated attribute levels at time of administration in a human clinical trial, levels of attribute exposure to patients of the clinical trial were estimated. Calculated fragment species of up to 15% based on a dose of 210 mg Q28D were not associated with any safety issues in vivo.

TABLE 7
Peak Area Percentage of Tezepelumab
Drug Substance by rCE-SDS

| Peak Identification | Relative Area % |
|---|---|
| LC + HC | 98.7 |
| LMW | <LOQ* |
| MMW | <LOQ* |
| NGHC | 0.6 |
| HMW | 0.4 |

*LOQ = 0.3%

CE-SDS can also be performed under non-reducing conditions in order to evaluate the presence of non-monomer species. This technique is performed under denaturing conditions to unfold the protein and disrupt non-covalent associations and is particularly useful for detection of partial molecule species and partially reduced intact molecules, i.e., those lacking one or more of the 2 light chain and 2 heavy chain constituents or respective inter-chain linkages expected of a monomeric antibody. Species consisting of 2 heavy chains associated with a single light chain (HHL) or a single heavy chain associated with a single light chain (HL, also known as half-molecule) have been reported from certain cell culture conditions (Trexler-Schmidt M, et al, 2010). Tezepelumab was denatured by heating in the presence of SDS and N-ethylmaleimide at pH 6.5 prior to electrokinetic injection into a bare-fused silica capillary (50 mm ID×30.2 cm) filled with SDS gel buffer at 25° C. Injection voltage was 10.0 kV, separation voltage was 15.0 kV, and absorbance was monitored at 220 nm. The data demonstrate that tezepelumab drug substance is predominantly composed of disulfide-linked heavy chain and light chain monomer, with low levels of smaller species comprising less than 4.5% of the distribution (Table 8).

TABLE 8
Peak Area Percentage of Tezepelumab
Drug Substance by nrCE-SDS

| Peak Identification | Relative Area % |
|---|---|
| Main Peak, HC + LC monomer | 95.5 |
| Pre-Peaks, smaller species | 4.5 |

Addition of static light scattering (SLS) detection to the SE-HPLC method allows the determination of molar mass for individual peaks in the chromatogram. The intensity of light scattered by an eluting species is proportional to both concentration and the molecular weight of the species. The intensity of UV absorbance (280 nm) is proportional to protein concentration. The molar mass of each eluting species can be determined by the instrument manufacturer's software by utilizing the light scattering intensity and concentration for each peak. Tezepelumab drug substance was analyzed by SE-HPLC chromatography coupled with on-line multi-angle light scattering detection using an Agilent 1100 HPLC system with a TSK-GEL G3000SWxA, 5 m particle size, 7.8 mm ID×300 mm length column. The detectors used were a Wyatt Heleos II detector, a Wyatt Optilab TrEX RI detector, and an Agilent UV detector with wavelength set at 280 nm. The SE-HPLC runs were performed at room temperature, with 100 mM sodium phosphate, 250 mM sodium chloride, pH 6.8±0.1 buffer used as the mobile phase and the flow rate was 0.5 mL/min.

The UV profile and corresponding molar masses calculated from SLS data generated for tezepelumab drug substance show that the molar mass of the main peak is 145 kDa, in close agreement with the theoretical mass of tezepelumab monomer (147 kDa). The molar mass for the peak eluting prior to monomer averages 284 kDa, in close agreement with the theoretical mass of tezepelumab dimer (294 kDa) indicating that the majority of HMW species are dimers of tezepelumab (Table 9).

TABLE 9
Molecular Weight of Main and Major HMW Peak of Tezepelumab
Drug Substance Determined by SE-HPLC-SLS

| Peak Identification | Molecular Weight (kDa)^{a} |
|---|---|
| Monomer | 145 ± 0.2 |
| Dimer (HMW) | 284 ± 6 |

The enriched HMW fraction (enriched for tezepelumab dimer) and main peak (containing primarily monomer) were assessed for potency by the receptor-ligand binding assay and cell-based reporter gene bioassay. The results show a reduction in potency as determined by receptor-ligand binding assay and cell-based reporter gene bioassay, at 64% and 62% of tezepelumab activity, respectively (Table 10).

TABLE 10
Potency Determination of SE-UHPLC Fractions

| | Receptor-ligand | Cell-based Reporter |
|---|---|---|
| | Binding Assay | Gene Bioassay |
| Sample Description | % Relative Potency | % Relative Potency |
| HMW | 64 | 62 |
| Main Peak | 108 | 96 |

This result is expected, as self-association imposes steric constraints, and may result in conformational changes, which in turn may affect binding. Increased rates of aggregate formation can occur under elevated temperature, low pH, physiological pH, visual and UV light exposure. The biological characterization determined that HMW species showed reduced potency. A reduction in the in vitro potency was only detectable when they were enriched to levels significantly exceeding the amounts detected under normal processing and storage conditions.

Disulfide Isoforms: Tezepelumab is an antibody of the IgG2 subclass and is therefore expected to display disulfide-mediated structural derivatives and isoforms (Wypych et al., Journal of Biological Chemistry, Vol. 283(23):16194-16205, 2008; Dillon et al., Journal of Biological Chemistry, Vol. 283(23):16206-16215, 2008). Disulfide structural heterogeneity is inherent to recombinant and naturally occurring IgG2 molecules, which contain 18 disulfide bonds—6 inter-chain and 12 intra-chain (Wang et al, 2007; Zhang and Czupryn, 2002). The connectivity of disulfide bonds detected in tezepelumab was elucidated using different approaches depending upon the number of linkages present in the non-reduced peptides. For peptides containing a single disulfide linkage, comparison of reduced and non-reduced tryptic peptide maps was used to assign disulfide connectivity.

Unlike the classical IgG2-A structure, IgG2-B isoform contain symmetrical linkages connecting two copies of the Fab peptides (C_{H}1-C_{L}-hinge) with two copies of the hinge peptide. IgG2-A/B represents an intermediate form, incorporating partial features of both IgG2-A and IgG2-B, defined by an asymmetrical arrangement involving one Fab arm covalently linked to two copies of the hinge peptide through disulfide bonds (Wypych et al., Journal of Biological Chemistry, Vol. 283(23):16194-16205, 2008; Dillon et al., Journal of Biological Chemistry, Vol. 283(23):16206-16215, 2008; Zhang et al., Anal Chem., Vol. 82(3):1090-1099, 2010).

Disulfide-linked peptides were identified in unfractionated drug substance by peptide mapping using endoprotease trypsin under non-reducing and reducing conditions. The outlet of the RP-HPLC separation was coupled to an electrospray ionization mass spectrometer (ESI-MS) for mass analysis in addition to UV detection. The non-reduced digest was subsequently treated with a reducing agent [tris(2-carboxyethyl) phosphine hydrochloride (TCEP)] and reanalyzed using the same conditions. Each disulfide-linked peptide from the non-reduced tryptic peptide map of tezepelumab drug substance was analyzed for its constituent peptides by mass spectrometry under reducing condition. Taken together, characterization of the disulfide-linked peptides designated A through H elucidated the linkages between specific Cys residues, summarized in Table 11, which confirm the presence of the classical disulfide structure, IgG2-A.

TABLE 11
Confirmed IgG2-A Connectivity for Peptides A through H

| Disulfide- | Constituent | Cys-Cys Bonds Identified | Loop |
|---|---|---|---|
| linked Peptide | Peptides | (Peptides) | (Element) |
| A | (H3)/(H12) | Cys²²(H3) - Cys⁹⁶(H12) | V_{H} (Fab) |
| B | (H15)/(H16) | Cys¹⁴⁹(H15) - Cys²⁰⁵(H16) | C_{H}1 (Fab) |
| C | (L2)/(L5) | Cys²²(L2) - Cys⁸⁷(L5) | V_{L} (Fab) |
| D | (L8)/(L14) | Cys¹³⁶(L8) - Cys¹⁹⁵(L14) | C_{L} (Fab) |
| E | (H14)/(L15) | Cys¹³⁶(H14) - Cys²¹³(L15) | Inter HC-LC |
| F | (H22)/(H27) | Cys²⁶²(H22) - Cys³²²(H27) | C_{H}2 (Fc) |
| G | (H35)/(H40) | Cys³⁶⁸(H35) - Cys⁴²⁶(H40) | C_{H}3 (Fc) |
| H | (H20)/(H20) | Cys²²⁴(H20) - Cys²²⁴(H20) | Hinge |
| | | Cys²²⁵(H20) - Cys²²⁵(H20) | |
| | | Cys²²⁸(H20) - Cys²²⁸(H20) | |
| | | Cys²³¹(H20) - Cys²³¹(H20) | |

The non-reduced tryptic peptide maps also showed the presence of IgG2-B derivative. Further confirmation of the IgG2-B disulfide derivative was done through non-reduced RP-HPLC. Taken together, characterization of the disulfide-linked peptides A through 0, peptides F through G, as well as peptide I elucidated the linkages between specific Cys residues, summarized in Table, which confirm the presence of the disulfide isoform structure, IgG2-B.

TABLE 12
Confirmed IgG2-B Connectivity for Peptides
A through D, F through G and I

| Disulfide- | Constituent | Cys-Cys Bonds Identified | Loop |
|---|---|---|---|
| linked Peptide | Peptides | (Peptides) | (Element) |
| A | (H3)/(H12) | Cys²²(H3) - Cys⁹⁶(H12) | V_{H} (Fab) |
| B | (H15)/(H16) | Cys¹⁴⁹(H15) - Cys²⁰⁵(H16) | C_{H}1 (Fab) |
| C | (L2)/(L5) | Cys²²(L2) - Cys⁸⁷(L5) | V_{L} (Fab) |
| D | (L8)/(L14) | Cys¹³⁶(L8) - Cys¹⁹⁵(L14) | C_{L} (Fab) |
| F | (H22)/(H27) | Cys²⁶²(H22) - Cys³²²(H27) | C_{H}2 (Fc) |
| G | (H35)/(H40) | Cys³⁶⁸(H35) - Cys⁴²⁶(H40) | C_{H}3 (Fc) |
| I | (H20)/(L15) | Cys²²⁴(H20) - Cys²¹³ (L15) | IgG2-B form |
| | (H14)/(H20) | Cys¹³⁶(H14) - Cys²²⁵(H20) | |
| | (H20)/(H20) | Cys²²⁸(H20) - Cys²²⁸(H20) | |
| | (H20)/(H20) | Cys²³¹(H20) - Cys²³¹(H20) | |

The presence of structural isoform IgG2-A/B was also found in the non-reduced tryptic peptide maps of tezepelumab. Further confirmation of IgG2-A/B disulfide isoform derivative is done through non-reduced RP-HPLC. Taken together, characterization of the disulfide-linked peptides A through G and peptide J elucidated the linkages between specific Cys residues, as summarized in Table, confirming the presence of the disulfide isoform structure, IgG2-A/B.

TABLE 13
Confirmed IgG2-A/B Connectivity for Peptides A through G, and J

| Disulfide- | Constituent | Cys-Cys Bonds Identified | Loop |
|---|---|---|---|
| linked Peptide | Peptides | (Peptides) | (Element) |
| A | (H3)/(H12) | Cys²²(H3) - Cys⁹⁶(H12) | V_{H} (Fab) |
| B | (H15)/(H16) | Cys¹⁴⁹(H15) - Cys²⁰⁵(H16) | C_{H}1 (Fab) |
| C | (L2)/(L5) | Cys²²(L2) - Cys⁸⁷(L5) | V_{L} (Fab) |
| D | (L8)/(L14) | Cys¹³⁶(L8) - Cys¹⁹⁵(L14) | C_{L} (Fab) |
| E | (H14)/(L15) | Cys¹³⁶(H14) - Cys²¹³(L15) | Inter HC-LC |
| F | (H22)/(H27) | Cys²⁶²(H22) - Cys³²²(H27) | C_{H}2 (Fc) |
| G | (H35)/(H40) | Cys³⁶⁸(H35) - Cys⁴²⁶(H40) | C_{H}3 (Fc) |
| J | (H14)/(H20) | Cys¹³⁶(H14) - Cys²²⁴(H20) | IgG2-A/B |
| | (H20)/(L15) | Cys²²⁴(H20) - Cys²¹³(L15) | form |
| | (H20)/(H20) | Cys²²⁵(H20) - Cys²²⁵(H20) | |
| | (H20)/(H20) | Cys²²⁸(H20) - Cys²²⁸(H20) | |
| | (H20)/(H20) | Cys²³¹(H20) - Cys²³¹(H20) | |

Comparison of reduced and non-reduced tryptic peptide mapping revealed the presence of expected disulfide-linked peptides for the predominant IgG2-A structure, as well as peptides bearing connectivity associated with the additional IgG2-A/B and IgG2-B disulfide structural isoforms. The relative level of disulfide isoforms in tezepelumab ranges from approximately 3.4-4.2% IgG2-B, 39.2-42% IgG2-A/B, and 54.2-57.1% IgG2-A based on RP-HPLC. Based on dosage and estimated attribute levels at time of administration in a human clinical trial, levels of attribute exposure to patients of the clinical trial were estimated. Calculated disulfide isoform derivatives IgG2-B of up to 15% based on a dose of 210 mg Q28D, and calculated disulfide isoform derivative IgG2-A/B of up to 75% based on a dose of 210 mg Q28D were not associated with any safety issues in vivo. The potency of the disulfide isoforms was evaluated by enriching the major isoforms in the drug substance, IgG2-A, IgG2-A/B, and IgG2-B using CEX-UHPLC. The results demonstrated that within assay capability, all isoforms showed full potency.

### Example 3—Leverage analysis of attributes and potency

Statistical analysis utilizing least square regression model was performed to assess relationship between HMW species, CDR isoAsp at D49D50, and total CDR oxidation. The analysis was based on these attributes as determined from the forced degradation analysis described in Example 1.

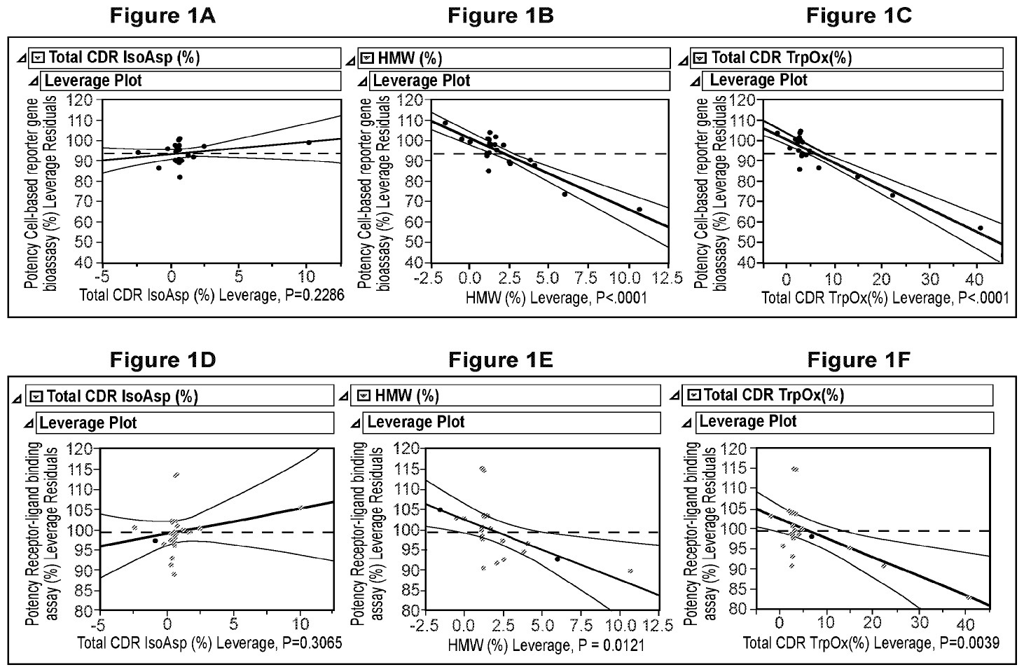

The analysis was performed with potency measurements using the Cell-Based Reporter Gene Bioassay described herein (FIGS. 1A-C) and also with potency measurements using the Receptor-Ligand Binding Assay described herein (FIGS. 1D-F). The identified relationships between attributes were comparable for both potency assays. Statistically significant negative correlations between HMW species and total CDR trp oxidation were identified (FIGS. 1B-C and 1E-F). Relationships between CDR IsoAsp D49D50 and potency did not reach statistical significance.

### Example 4—High Mannose species and pharmacokinetic (PK) modeling

A modeling approach was used to estimate the potential impact of an increase in % high mannose (HM) on the clearance of tezepelumab. The model assumed a half-life of 24.5 days (PK profile from IV doses of tezepelumab in a clinical study) and an increased rate constant of 0.035 Day-1 for the HM form of tezepelumab based on the HM % decrease rate of a reference IgG2 monoclonal antibody following single IV dose. The increased rate constant of a reference IgG2 monoclonal antibody HM form has the highest value analyzed to date and was chosen as a conservative estimate of the tezepelumab HM half-life. The PK profile was modeled up to 122.5 days (the equivalent of 5 half-lives) and no correction for preferential pairing was used. As shown in Table 14 below, a relationship was modeled between HM levels and estimated increase in clearance of tezepelumab.

TABLE 14
High Mannose levels and estimated impact on clearance

| HM Levels | Estimated Increase in the Clearance |
|---|---|
| 5% | N/A |
| 8% | 1.7% |
| 11% | 3.3% |
| 13% | 4.4% |
| 15% | 5.5% |
| 18% | 7.2% |
| 21% | 9.4% |
| 23.1% | 10.0% |

These results indicate that the tezepelumab composition having 5% or less HM species showed little to no increase in clearance, and approximately 23% HM species in a tezepelumab composition resulted in approximately a 10% increase in antibody clearance.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain many equivalents to the specific embodiments of the invention described herein. Such equivalents are intended to be encompassed by the following claims.

What is claimed:

1. A composition comprising tezepelumab and one or more tezepelumab derivatives, wherein the one or more tezepelumab derivatives comprise an isomerization derivative, and wherein the amount of the isomerization derivative in the composition is less than about 30%, wherein tezepelumab comprises
A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7 and
(iii) a heavy chain CDR3 amino acid sequence set out in SEQ ID NO:8.

2. The composition of claim 1, wherein the amount of the isomerization derivative in the composition is from about 0.5% to about 13%.

3. The composition of claim 1 or 2, wherein the isomerization derivative comprises a modification in the heavy chain or light chain complementarity determining region (CDR).

4. The composition of any one of claims 1 to 3, wherein the isomerization derivative comprises a change at heavy chain CDR D54 of SEQ ID NO: 7, and/or light chain CDR D49, D50 or D52 of SEQ ID NO: 4 in either or both variable region chains.

5. The composition of any one of claims 1 to 4, wherein the isomerization derivative comprises isomerization at D54 of SEQ ID NO: 7 in an amount of less than about 5%.

6. The composition of any one of claims 1 to 4, wherein the isomerization derivative comprises isomerization at one or more of D49, D50 or D52 of SEQ ID NO: 4 in an amount of less than about 13%.

7. The composition of any one of claims 1 to 6, wherein the isomerization derivative is isoaspartic acid (isoAsp) or cyclic aspartate (cAsp).

8. The composition of any one of claims 1 to 7, wherein the amount of the isomerization derivative in the composition is determined by reduced peptide mapping.

9. The composition of any one of claims 1 to 8, wherein the tezepelumab and tezepelumab derivatives have a greater potency and/or tolerability than a composition comprising greater than 30% of the isomerization derivative, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR.

10. A composition comprising tezepelumab and one or more tezepelumab derivatives, wherein the one or more tezepelumab derivatives comprise a deamidation derivative, and wherein the amount of the deamidation derivative in the composition is less than about 15%, wherein tezepelumab comprises
A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7 and
(iii) a heavy chain CDR3 amino acid sequence set out in SEQ ID NO:8.

11. The composition of claim 10, wherein the amount of the deamidation derivative in the composition is between about 0.5%-10%.

12. The composition of claim 10 or 11 wherein the deamidation derivative comprises deamidated asparagine N25/N26 of SEQ ID NO: 3, N316 of SEQ ID NO: 13, and/or N385/390 of SEQ ID NO: 13.

13. The composition of any one of claims 10 to 12, wherein the deamidation derivative comprises deamidation at N25/N26 of SEQ ID NO: 3 in an amount of less than about 3%.

14. The composition of any one of claims 10 to 12, wherein the deamidation derivative comprises deamidation at one or more of N316, and/or N385/390 of SEQ ID NO: 13 in an amount of less than about 13%.

15. The composition of any one of claims 10 to 14, wherein the amount of the deamidation derivative in the composition is determined by reduced peptide mapping.

16. The composition of any one of claims 10 to 15, wherein the tezepelumab and tezepelumab derivatives have a greater potency and/or tolerability than a composition comprising greater than 15% of the deamidation derivative, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR.

17. A composition comprising tezepelumab and one or more tezepelumab derivatives, wherein the one or more tezepelumab derivatives comprises an oxidation derivative, and wherein the amount of the oxidation derivative in the composition is less than about 7%, wherein tezepelumab comprises
A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7 and
(iii) a heavy chain CDR3 amino acid sequence set out in SEQ ID NO:8.

18. The composition of claim 17, wherein the amount of the oxidation derivative in the composition is between about 0.4% to about 7%.

19. The composition of claim 17 or 18, wherein the oxidation derivative comprises oxidation at one or more of heavy chain methionine M34 of SEQ ID NO: 6, M253, M359 of SEQ ID NO: 13, or heavy chain tryptophan W52 of SEQ ID NO: 7, W90 of SEQ ID NO: 5, or W102 of SEQ ID NO: 8, in either or both heavy chains.

20. The composition of any one of claims 17 to 19, wherein the oxidation derivative comprises oxidation at one or more of heavy chain methionine M34 of SEQ ID NO: 6, M253, M359 of SEQ ID NO: 13, in either or both heavy chains, optionally wherein the oxidation is in an amount of less than about 7%.

21. The composition of any one of claims 17 to 19, wherein the oxidation derivative comprises oxidation at one or more of tryptophan W52 of SEQ ID NO: 7, W90 of SEQ ID NO: 5, or W102 of SEQ ID NO: 8 in either or both heavy chains, optionally wherein the oxidation is in an amount of less than about 3%.

22. The composition of any one of claims 17 to 21, wherein the amount of the oxidation derivative in the composition is determined by reduced peptide mapping.

23. The composition of any one of claims 17 to 22, wherein the tezepelumab and tezepelumab derivatives have a greater potency and/or tolerability than a composition comprising greater than 7% of the oxidation derivative, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR.

24. A composition comprising tezepelumab and one or more tezepelumab derivatives, wherein the one or more tezepelumab derivatives are high molecular weight (HMW) species, and wherein the amount of the HMW species in the composition is less than about 20%, wherein tezepelumab comprises
A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7; and
(iii) a heavy chain CDR3 amino acid sequence set out in SEQ ID NO:8.

25. The composition of claim 24, wherein the amount of the HMW species in the composition is about 1.7% or less.

26. The composition of claim 24 or 25, wherein the amount of the HMW species in the composition is about 1.4% or less.

27. The composition of any one of claims 24 to 26, wherein the HMW species comprises a dimer of tezepelumab.

28. The composition of any one of claims 24 to 27, wherein the amount of the HMW species in the composition is determined by size exclusion-high performance liquid chromatography (SE-HPLC).

29. The composition of claim 28 wherein the SE-HPLC is SE-Ultra HPLC wherein the proteins are separated isocratically using a mobile phase comprising 100 mM sodium phosphate, 250 mM sodium chloride at pH 6.8.

30. The composition of any one of claims 24 to 29, wherein the tezepelumab and tezepelumab derivatives have a greater potency and/or tolerability than a composition comprising greater than 20% of the HWM species, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR.

31. A composition comprising tezepelumab and one or more tezepelumab derivatives, wherein the one or more tezepelumab derivatives comprise a tezepelumab fragment, and wherein the amount of the tezepelumab fragment in the composition is less than about 15%, wherein tezepelumab comprises
(A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7 and
(iii) a heavy chain CDR3 amino acid sequence set out in SEQ ID NO:8.

32. The composition of claim 31, wherein the tezepelumab fragments are low molecular weight (LMW) or middle molecular weight (MMW) species, or combinations thereof.

33. The composition of claim 31 or 32, wherein the fragments are low molecular weight species of less than about 25 kD.

34. The composition of claim 31 or 32, wherein the fragments are middle molecular weight species having a molecular weight between about 25 to 50 kD.

35. The composition of any one of claims 31 to 34, wherein the amount of tezepelumab fragment in the composition is determined by reduced capillary electrophoresis with sodium dodecyl sulfate (rCE-SDS).

36. The composition of any one of claims 31 to 35, wherein the tezepelumab and tezepelumab derivatives have a greater potency and/or tolerability than a composition comprising greater than 15% of the tezepelumab fragments, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR.

37. A composition comprising tezepelumab and one or more tezepelumab derivatives, wherein the one or more tezepelumab derivatives comprises a glycosylation derivative, and wherein the amount of the glycosylation derivative in the composition is less than about 40%, wherein tezepelumab comprises
(A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7 and
(iii) a heavy chain CDR3 amino acid sequence set out in SEQ ID NO:8.

38. The composition of claim 37, wherein the amount of glycosylation derivative in the composition is less than about 35%, about 30%, about 25%, about 20%, about 15%, about 10% or about 5%.

39. The composition of claim 37 or 38, wherein the glycosylation derivative comprises alteration of tezepelumab glycosylation on residue N298 of SEQ ID NO: 13, on one or both heavy chains.

40. The composition of any one of claims 37 to 39, wherein the glycosylation derivative comprises afucosylation or alteration of glycosylation of tezepelumab to high mannose moieties or galactosyl moieties.

41. The composition of any one of claims 37 to 40, wherein the glycosylation derivative comprises afucosylated derivatives in an amount of less than about 5%.

42. The composition of any one of claims 37 to 40, wherein the glycosylation derivative comprises galactosyl moieties in an amount of less than about 30%.

43. The composition of any one of claims 37 to 40, wherein the glycosylation derivative comprises high mannose moieties in an amount of less than about 5%.

44. The composition of any one of claims 37 to 40 or 43, wherein the tezepelumab and tezepelumab derivatives comprise no more than about 25%, about 23%, about 21%, about 19%, about 17%, about 15%, about 13%, about 11%, about 8%, or about 5% high mannose glycosylation derivatives.

45. The composition of any one of claims 37 to 40, 43 or 43A, wherein the tezepelumab and tezepelumab derivatives have less clearance and/or a longer half-life than a composition having greater than 25% high mannose glycosylation derivatives.

46. The composition of any one of claims 37 to 45, wherein the amount of the glycosylation derivative in the composition is determined by glycan map method.

47. The composition of any one of claims 37 to 46, wherein
(a) the tezepelumab and tezepelumab derivatives have a greater potency and/or tolerability than a composition comprising greater than 40% of the glycosylation derivatives, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR; or
(b) the tezepelumab and tezepelumab derivatives comprise no more than 15% high mannose, and have less clearance than a composition having greater than 15% high mannose.

48. The composition of any one of claims 37 to 46, wherein
(a) the tezepelumab and tezepelumab derivatives have a greater potency and/or tolerability than a composition comprising greater than 40% of the glycosylation derivatives, wherein said potency comprises a capability of inhibiting binding of biotinylated TSLPR immobilized on a donor bead to TSLP-His immobilized on an acceptor bead or a capability of inhibiting binding of TSLPR expressed on the surface of a Stat/BaF/HTR cell encoding a Stat luciferase reporter gene, the expression of which is indicative of binding of TSLP to TSLPR; or
(b) the tezepelumab and tezepelumab derivatives comprise no more than 25% high mannose, and have less clearance than a composition having greater than 25% high mannose.

49. A composition comprising tezepelumab and one or more disulfide isoform derivatives thereof, wherein the one or more disulfide isoform derivatives comprise an IgG2-B isoform and or an IgG2-A/B isoform, and wherein the amount of the disulfide isoform derivative in the composition is less than about 75%.

50. The composition of claim 49 wherein the one or more disulfide isoform derivatives comprise an IgG2-B isoform, and wherein the amount of the disulfide derivative in the composition is less than about 20%.

51. The composition of claim 50, wherein the amount of the IgG2-B isoform is less than about 5%.

52. The composition of claim 49, wherein the one or more disulfide isoform derivatives comprise an IgG2-A/B isoform.

53. The composition of claim 52, wherein the amount of the IgG2-A/B isoform in the composition is less than about 75%.

54. The composition of claim 52, wherein the amount of the IgG2-A/B isoform in the composition is from about 38% to about 43%.

55. The composition of any one of claims 48 to 54, wherein the amount of disulfide isoform derivatives in the composition is determined by non-reduced reversed phase high performance liquid chromatography (RP-HPLC).

56. A composition comprising tezepelumab and one or more tezepelumab derivatives, wherein the tezepelumab derivatives comprise isomerization derivatives, deamidation derivatives, oxidation derivatives, glycosylation derivatives, HMW species, fragments, disulfide isoform derivatives or combinations thereof, wherein the composition has one or more of the following characteristics:
(a) the amount of isomerization derivatives in the composition is about 30% or less as measured by reduced peptide mapping;
(b) the amount of deamidation derivatives in the composition is about 15% or less as measured by peptide mapping;
(c) the amount of oxidation derivatives in the composition is about 7% or less as measured by reduced peptide mapping;
(d) the amount of glycosylation derivatives in the composition is about 40% or less as measured by glycan mapping;
(e) the amount of disulfide isoform derivatives in the composition is about 75% or less as measured by non-reduced reversed phase high performance liquid chromatography (RP-HPLC);
(f) the amount of HMW species in the composition is about 20% or less as measured by SE-HPLC; and/or
(g) the amount of fragments in the composition is about 15% or less as measured by rCE-SDS.

57. The composition of any one of claims 49 to 56, wherein tezepelumab comprises
(A) a light chain variable domain comprising:
(i) a light chain CDR1 amino acid sequence set out in SEQ ID NO:3;
(ii) a light chain CDR2 amino acid sequence set out in SEQ ID NO: 4; and
(iii) a light chain CDR3 amino acid sequence set out in SEQ ID NO:5; and
(B) a heavy chain variable domain comprising:
(i) a heavy chain CDR1 amino acid sequence set out in SEQ ID NO:6;
(ii) a heavy chain CDR2 amino acid sequence set out in SEQ ID NO:7 and
(iii) a heavy chain CDR3 amino acid sequence set out in SEQ ID NO:8.

58. The composition of any one of claims 1 to 57, wherein tezepelumab comprises a heavy chain amino acid sequence set out in SEQ ID NO: 10 and a light chain amino acid sequence set out in SEQ ID NO: 12.

59. A pharmaceutical formulation comprising the composition of any one of claims 1 to 58 and one or more pharmaceutically acceptable excipients.

60. A method for treating an inflammatory disease in a subject comprising administering to the subject a therapeutically effective amount of a composition of any one of claims 1 to 58 or the pharmaceutical formulation of claim 59.

61. The method of claim 60, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

62. The method of claim 60 or 61, comprising administering the composition at an interval of every 2 weeks or every 4 weeks.

63. The method of any one of claims 60 to 62, wherein the composition is administered for a period of at least 4 months, 6 months, 9 months, 1 year or more.

64. The method of any one of claims 60 to 63, wherein the asthma is severe asthma.

65. The method of any one of claims 60 to 64, wherein the asthma is eosinophilic or non-eosinophilic asthma.

66. The method of any one of claims 60-65, wherein the administration is via pre-filled syringe or autoinjector.

67. The method of claim 66 wherein the auto-injector is an Ypsomed YpsoMate® device.

68. A tezepelumab composition of any one of claims 1 to 58 or a pharmaceutical composition of claim 59 for use in treating an inflammatory disease in a subject.

69. The composition of claim 68, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

70. Use of a tezepelumab composition of any one of claims 1 to 58 or a pharmaceutical composition of claim 59 in the preparation of a medicament for treating an inflammatory disease in a subject.

71. The composition or use of any one of claims 68 to 70, wherein the administration is via pre-filled syringe or autoinjector.

72. The composition or use of claim 71, wherein the auto-injector is an Ypsomed YpsoMate® device.

73. The composition or use of any one of claims 68 to 72, wherein the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease, IgA nephropathy, lupus nephritis, eosinophilic gastritis, chronic sinusitis without nasal polyps and idiopathic pulmonary fibrosis (IPF).

74. A method for assessing the quality of a tezepelumab composition, comprising:
obtaining a tezepelumab composition that contains tezepelumab and one or more tezepelumab derivatives;
measuring the amount of one or more tezepelumab derivatives in the composition, wherein the tezepelumab derivatives comprise isomerization derivatives, deamidation derivatives, oxidation derivatives, glycosylation derivatives, disulfide isoform derivatives, HMW species, fragments, or combinations thereof;
comparing the measured amount of the one or more tezepelumab derivatives to a pre-determined reference criterion; and
preparing a pharmaceutical formulation or pharmaceutical product of the tezepelumab composition if the comparison indicates that the pre-determined reference criterion is met.

75. The method of claim 74, wherein the amount of isomerization derivatives is measured and the pre-determined reference criterion is about 30% or less.

76. The method of claim 74 or 75, wherein the amount of isomerization in the tezepelumab composition is measured by reduced peptide mapping.

77. The method of claim 74, wherein the amount of deamidation derivatives is measured and the pre-determined reference criterion is about 15% or less.

78. The method of claim 74 or 77, wherein the amount of deamidation in the tezepelumab composition is measured by reduced peptide mapping.

79. The method of claim 74, wherein the amount of oxidation derivatives is measured and the pre-determined reference criterion is about 7% or less.

80. The method of claim 74 or 79, wherein the amount of oxidation in the tezepelumab composition is measured by reduced peptide mapping.

81. The method of claim 74, wherein the amount of glycosylation derivatives is measured and the pre-determined reference criterion is about 40% or less.

82. The method of claim 74 or 81, wherein the amount of glycosylation in the tezepelumab composition is measured by glycan mapping.

83. The method of claim 74, wherein the amount of disulfide isoform derivatives is measured and the pre-determined reference criterion is about 75% or less.

84. The method of claim 74 or 83, wherein the amount of disulfide isoform in the tezepelumab composition is measured by non-reduced reversed phase high performance liquid chromatography (RP-HPLC).

85. The method of claim 74, wherein the amount of HMW species is measured and the pre-determined reference criterion is about 20% or less.

86. The method of claim 74 or 85, wherein the amount of HMW species is measured by SE-HPLC.

87. The method of claim 74, wherein the amount of fragments is measured and the pre-determined reference criterion is about 15% or less.

88. The method of claim 74 or 87, wherein the amount of fragments in the tezepelumab composition is measured by rCE-SDS.

89. The method of any one of claims 74 to 88, wherein the tezepelumab composition is obtained from a Chinese Hamster Ovary (CHO) cell line that expresses a nucleic acid encoding a heavy chain of SEQ ID NO: 10 and a nucleic acid encoding a light chain of SEQ ID NO: 12.


SEQUENCE LISTING (14 sequences)

SEQ ID NO: 1 - DNA, 743 bp, Homo Sapiens
  misc_feature: TSLP
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo Sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo Sapiens
  MISC_FEATURE: LCDR1
GGNNLGSKSVH

SEQ ID NO: 4 - protein, 7 aa, Homo Sapiens
  MISC_FEATURE: LCDR2
DDSDRPS

SEQ ID NO: 5 - protein, 11 aa, Homo Sapiens
  MISC_FEATURE: LCDR3
QVWDSSSDHVV

SEQ ID NO: 6 - protein, 5 aa, Homo Sapiens
  MISC_FEATURE: HCDR1
TYGMH

SEQ ID NO: 7 - protein, 17 aa, Homo Sapiens
  MISC_FEATURE: HCDR2
VIWYDGSNKHYADSVKG

SEQ ID NO: 8 - protein, 13 aa, Homo Sapiens
  MISC_FEATURE: HCDR3
APQWELVHEAFDI

SEQ ID NO: 9 - DNA, 366 bp, Homo Sapiens
  misc_feature: Heavy Chain VH
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo Sapiens
  MISC_FEATURE: Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo Sapiens
  misc_feature: Light Chain VL
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo Sapiens
  MISC_FEATURE: Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 448 aa, Artificial Sequence
  Synthetic Polypeptide
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 214 aa, Artificial Sequence
  Synthetic Polypeptide
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS